(19) United States
(12) Patent Application Publication
(10) Pub. No.: US 2012/0142718 A1
(43) Pub. Date: Jun. 7, 2012

(54) N-17-Alkylated Prodrugs of Opioids

(76) Inventors: Thomas E. Jenkins, Half Moon Bay, CA (US); Aleksandr Kolesnikov, San Francisco, CA (US)

(21) Appl. No.: 12/524,523

(22) PCT Filed: Feb. 15, 2008
(86) PCT No.: PCT/US08/54144

§ 371 Date: Apr. 16, 2010

Related U.S. Application Data
(60) Provisional application No. 60/901,795, filed on Feb. 16, 2007.
(60) Provisional application No. 60/990,193, filed on Nov. 26, 2007.

(51) Int. Cl. A61K 31/46; C07D 489/02

(52) U.S. Cl. 514/282; 546/45

(57) ABSTRACT

Compounds of formula (II) R₁R₂R₃N⁺—(C(R^{1a})(R^{2a}))—Ar—Z—C(O)—Y—(C(R¹)(R²))ₙ—N—(R³)(R⁴)A⁻II, in which R₁R₂R₃N⁺, R^{1a}, R^{2a}, Ar, Z, Y, R¹, R², n, R³, R⁴ and A⁻ have the meanings given in the specification, are useful as prodrugs for opioids (Example 13).

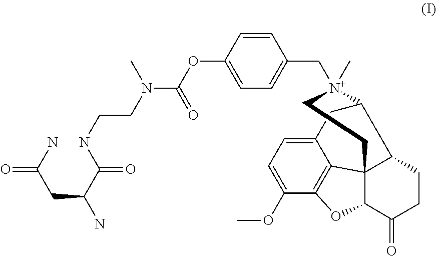

Disclosed herein are prodrugs of opioids. Also disclosed herein are methods of making prodrugs of opioids, pharmaceutical compositions of prodrugs of opioids and methods of using prodrugs of opioids and pharmaceutical compositions thereof to treat or prevent various diseases.

Delivery systems are often essential in safely administering active agents such as drugs, which contain the phenol, thiol, or aniline functionality. Often delivery systems can optimize bioavailability, improve dosage consistency and improve patient compliance (e.g., by reducing dosing frequency). Solutions to drug delivery and/or bioavailability issues in pharmaceutical development include converting known drugs to prodrugs. Typically, in a prodrug, a polar functional group (e.g., a carboxylic acid, an amino group, phenol group, a sulfhydryl group, etc.) of the active agent is masked by a promoiety, which is labile under physiological conditions. Accordingly, prodrugs are usually transported through hydrophobic biological barriers such as membranes and may possess superior physicochemical properties in comparison to the parent drug. Prodrugs are usually non-toxic and are ideally selectively cleaved at the locus of drug action. Preferably, cleavage of the promoiety occurs rapidly and quantitatively with the formation of non-toxic by-products (i.e., the hydrolyzed promoiety).

Prodrugs as described above are capable of providing patients with safe and effective treatment if the patients follow the directions given by the attending physician. Unfortunately human patients do not always follow the directions that they have been given. They may accidentally take an overdose of the prodrug, or deliberately abuse it, for example by taking an overdose, by injecting or inhaling it, or by using readily available household chemicals (like vinegar or baking soda) to obtain the active drug from the prodrug. Abuse is a particular concern with prodrugs of recreational or addictive drugs, like amphetamines and opioids.

It would be desirable to have a prodrug that has built-in control, so that it is difficult to use the prodrug other than in the way it is intended.

A new way has now been found for configuring prodrugs of opioids that affords controlled release of the drugs.

According to one aspect, the present invention provides a method of providing a patient with post administration-activated, controlled release of an opioid, which comprises administering to said patient a corresponding compound of formula (II)

R¹R²R³N+—(C(R^{1a})(R^{2a})(R^{2a}))—Ar—Z—C(O)—Y—(C(R)(R²))ₙ—N—(R³)(R⁴)A⁻ II

or a salt, hydrate or solvate thereof wherein:

R₁R₂R₃N⁺—represents a residue of an opioid wherein the lone pair of electrons of the tertiary amine nitrogen atom is replaced with a bond to —(C(R^{1a})(R^{2a})—Ar—Z—C(O)—Y—(C(R¹)(R²))ₙ—N—(R³)(R⁴);

R^{1a} and R^{2a} are independently hydrogen, alkyl, substituted alkyl, alkoxy, substituted alkoxy, aryl, substituted aryl, arylalkyl, substituted arylalkyl, heteroaryl, substituted heteroaryl, heteroarylalkyl or substituted heteroarylalkyl;

Ar is aryl, heteroaryl or arylaryl optionally substituted with one or more —F, —Cl, —Br, —I, —R^{4a}, —O⁻, —OR^{4a}, —SR^{4a}, —S⁻, —NR^{4a}R^{5a}, —CF₃, —CN, —OCN, —SCN, —NO, —NO₂, —N₃, —S(O)₂O⁻, —S(O)₂OH, —S(O)₂R^{4a}, —OS(O₂)O⁻, —OS(O)₂R^{4a}, —P(O)(O⁻)₂, —P(O)(OR^{4a})(O⁻), —OP(O)(OR^{4a})(OR^{5a}), —C(O)R^{4a}, —C(S)R^{4a}, —C(O)OR^{4a}, —C(O)NR^{4a}R^{5a}, —C(O)O, —C(S)OR^{4a}, —NR^{6a}C(O)NR^{4a}R^{5a}, NR^{6a}C(S)NR^{4a}R^{5a}NR^{7a}C(NR^{6a})NR^{5a}R^{4a} or —C(NR^{6a})NR^{5a}R^{4a}, or tethered to a polymer;

R^{4a}, R^{5a}, R^{6a} and R^{7a} are independently hydrogen, alkyl, substituted alkyl, cycloalkyl, substituted cycloalkyl, cycloheteroalkyl, substituted cycloheteroalkyl, aryl, substituted aryl, heteroaryl or substituted heteroaryl, or optionally R⁴ and R⁵ together with the nitrogen atom to which they are bonded form a cycloheteroalkyl or substituted cycloheteroalkyl ring;

Z is O, S or NH;

Y is —NR⁵—, —O— or —S—;

n is an integer from 1 to 10;

each R¹, R², R³ and R⁵ is independently hydrogen, alkyl, substituted alkyl, aryl or substituted aryl, or R¹ and R² together with the carbon to which they are attached form a cycloalkyl or substituted cycloalkyl group, or two R¹ or R² groups on adjacent carbon atoms, together with the carbon atoms to which they are attached, form a cycloalkyl or substituted cycloalkyl group;

R⁴ is

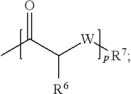

each R⁶ is independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, or optionally, R⁶ and R⁷ together with the atoms to which they are bonded form a cycloheteroalkyl or substituted cycloheteroalkyl ring;

R⁷ is hydrogen, alkyl, substituted alkyl, acyl, substituted acyl, alkoxycarbonyl, substituted alkoxycarbonyl, aryl, substituted aryl, arylalkyl or substituted arylalkyl;

p is an integer from 1 to 5;

each W is independently —NR⁸—, —O— or —S—;

each R⁸ is independently hydrogen, alkyl, substituted alkyl, aryl or substituted aryl, or optionally, each R⁶ and R⁸ independently together with the atoms to which they are bonded form a cycloheteroalkyl or substituted cycloheteroalkyl ring; and

A⁻ represents an anion.

In the compounds of formula (II), the tertiary amine in an opioid has been substituted with a spacer group bearing a nitrogen nucleophile that is protected with an enzymatically-cleavable moiety (R⁴), the configuration of the spacer leaving group and nitrogen nucleophile being such that, upon enzymatic cleavage of the cleavable moiety, the nitrogen nucleophile is capable of liberating the compound from the spacer leaving group so as to provide the patient with controlled release of the compound.

In another aspect, the present invention provides the use in the manufacture of a medicament for providing a patient with post administration-activated, controlled release of a compound of formula (II) as defined hereinabove.

The corresponding compound (prodrug in accordance with the present invention) provides post administration-activated, controlled release of the compound, because it requires enzymatic cleavage (of the group R⁴) to initiate release of the compound, and because the rate of release of the compound depends upon both the rate of enzymatic cleavage and the rate of cyclisation. Accordingly, the prodrug has reduced susceptibility to accidental overdosing or abuse, whether by deliberate overdosing, administration through an inappropriate route, such as by injection, or by chemical modification using readily available household chemicals. The prodrug is configured so that it will not provide excessively high plasma levels of the active drug if it is administered inappropriately, and cannot readily be decomposed to afford the active drug other than by enzymatic-cleavage.

The enzyme capable of cleaving the enzymatically-cleavable moiety may be a peptidase—the enzymatically-cleavable moiety being linked to the nucleophilic nitrogen through an amide (e.g. a peptide bond: —NHCO—). In some embodiments, the enzyme is a digestive enzyme such as, for example, pepsin, trypsin, chymotrypsin, colipase, elastase, aminopeptidase N, aminopeptidase A, dipeptidylaminopeptidase IV, tripeptidase or enteropeptidase. Accordingly, in one embodiment of the method, the corresponding compound is administered orally to the patient.

The enzyme-cleavable moiety linked to the nitrogen nucleophile through an amide bond (the group R⁴) may be, for example, a residue of an amino acid or a peptide, or an N-acyl derivative of an amino acid or peptide (for example an N-acyl derivative of a pharmaceutically acceptable carboxylic acid, such as an N-acetyl derivative). The peptide may contain, for example, up to 10 amino acid residues. For example, it may be a dipeptide or tripeptide. Each amino acid may advantageously be a naturally occurring D or L-amino acid (such as an L-amino acid). Alternatively, one or more of the amino acids may be an unnatural amino acid that can be cleaved by an enzyme that cleaves enzyme-cleavable moieties. Examples of naturally occurring amino acids are alanine, arginine, asparagine, aspartic acid, cysteine, glycine, glutamine, glutamic acid, histidine, isoleucine, leucine, methionine, phenylalanine, proline, serine, threonine, tryptophan, tyrosine, lysine and valine. Accordingly, examples of enzyme-cleavable moieties include residues of the L-amino acids listed hereinabove and the N-acetyl derivatives thereof, and dipeptides and tripeptides formed from two or three of the L-amino acids listed hereinabove, and the N-acetyl derivatives thereof.

The cyclic group formed when the compound is released is conveniently pharmaceutically acceptable, in particular a pharmaceutically acceptable cyclic urea, carbamate or thiocarbamate. It will be appreciated that cyclic ureas in particular are generally very stable and have low toxicity.

It will be appreciated that when W is NH and R⁷ is H or acyl, then R⁴ is a residue of an amino acid or peptide, or an N-acyl derivative thereof.

Accordingly, in another embodiment, R⁴ is a residue of a D or L-amino acid (such as an L-amino acid) selected from alanine, arginine, asparagine, aspartic acid, cysteine, glycine, glutamine, glutamic acid, histidine, isoleucine, leucine, methionine, phenylalanine, proline, serine, threonine, tryptophan, tyrosine, lysine and valine; a residue of a dipeptide or tripeptide composed of two or three L-amino acid residues selected independently from alanine, arginine, asparagine, aspartic acid, cysteine, glycine, glutamine, glutamic acid, histidine, isoleucine, leucine, methionine, phenylalanine, proline, serine, threonine, tryptophan, tyrosine, lysine and valine; or a residue of an N-acyl derivative thereof, such as an N-acetyl derivative.

In one embodiment, each of R¹, R², R³ and R⁵ is independently hydrogen, alkyl, substituted alkyl, aryl or substituted aryl.

In another embodiment, R⁶ is a side atom or group of a natural amino acid, such as H (from glycine), —CH₂ (CH₂)₃NH₂ (from lysine), —CH₂CH₂CH₂NHC(NH)NH₂ (from arginine), —CH₃ (from alanine), —CH₂CH(CH₃)₂ (from leucine), —CH₂C(═O)NH₂ (from asparagine), —CH₂COOH (from aspartic acid), —CH₂ (p-hydroxyphenyl) (from tyrosine), or CH₂CH₂COOH (from glutamic acid).

In another embodiment, R⁷ is a hydrogen atom, or an unsubstituted or substituted acyl group, for example (1-6C)alkanoyl, such as acetyl or t-butanoyl; benzoyl unsubstituted or substituted by methylenedioxy or one or two substituents selected from (1-4C)alkyl, (1-4C)alkoxy or halogen, such as benzoyl or piperonyl; CONRₓR^{y} in which Rₓ and R_{y} are each independently hydrogen or (1-4C)alkyl, such as CONH₂), or a hemiacid or hemiester, such as CH₂CH₂COOH or CH₂CH₂COOEt. The unsubstituted of substituted acyl group is conveniently the residue of a pharmaceutically acceptable carboxylic acid. Examples of particular values for R⁷ are a hydrogen atom and acetyl and benzoyl.

It will be appreciated that when the N—R⁴ amide bond is cleaved enzymatically, the nitrogen nucleophile is freed and cyclises back onto the carbonyl group, forming the cyclic urea and releasing the compound, but this released compound undergoes a spontaneous 1,6-elimination to release another compound of formula RH (the opioid). For example, when R^{1a} and R^{2a} each represents hydrogen and Z represents O, the elimination reaction affords a compound of formula RH and a compound of formula

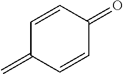

In one embodiment, Ar represents an unsubstituted or substituted 1,2-phenylene or 1,4-phenylene group, for example a 1,2-phenylene or 1,4-phenylene group that is unsubstituted or substituted by one or two substituents selected independently from a halogen atom (for example fluorine or chlorine); a (1-4C)alkyl group, such as methyl; a (1-4C)alkoxy group, such as methoxy; a carboxy group; or a hydroxy(1-4C)alkyl group, such as hydroxymethyl. It will be appreciated by those skilled in the art that an ortho carboxy or hydroxy(1-4C)alkyl substituent can function as an internal nucleophile to form a ring in the elimination step.

In one embodiment, R⁴ is a residue of a D- or L-amino acid (for example a residue of an L-amino acid) selected from alanine, arginine, asparagine, aspartic acid, cysteine, glycine, glutamine, glutamic acid, histidine, isoleucine, leucine, methionine, phenylalanine, proline, serine, threonine, tryptophan, tyrosine, lysine and valine; a residue of a dipeptide or tripeptide composed of two or three L-amino acid residues selected independently from alanine, arginine, asparagine, aspartic acid, cysteine, glycine, glutamine, glutamic acid, histidine, isoleucine, leucine, methionine, phenylalanine, proline, serine, threonine, tryptophan, tyrosine, lysine and valine; or a residue of an N-acyl derivative thereof, such as an N-acetyl derivative.

Examples of particular values are:

for R^{1a} and R^{2a}: hydrogen;

### for Z: O;

for Ar: 1,4-phenylene;
for R: hydrocodone, oxycodone, oxymorphone or hydromorphone residue

(such as hydrocodone, oxymorphone or hydromorphone, for example oxymorphone or hydromorphone);

### for A⁻: Cl⁻;

### for Y: —NR⁵;

for R⁵: (1-4C)alkyl, such as —CH₃;
for R¹ and R²: hydrogen or (1-4C)alkyl, such as CH₃; more particularly hydrogen;
for n: 2 or 3;
for R³: hydrogen or (1-4C)alkyl, such as —CH₃;

### for W: NH;

for R⁶: a side atom or group of a natural amino acid, such as H (from glycine), —CH₃ (from alanine), —CH₂CH(CH₃)₂ (from leucine), —CH₂ (CH₂)₃NH₂ (from lysine), —CH₂CH₂CH₂NHC(NH)NH₂ (from arginine), —CH₂C(═O)NH₂ (from asparagine), —CH₂COOH (from aspartic acid), —CH₂ (p-hydroxyphenyl) (from tyrosine) or CH₂CH₂COOH (from glutamic acid);
for R⁷: hydrogen, (1-6C)alkanoyl, such as acetyl or t-butanoyl, or optionally substituted benzoyl, for example benzoyl unsubstituted or substituted by methylenedioxy or one or two substituents selected from (1-4C)alkyl, (1-4C)alkoxy or halogen, such as benzoyl or piperonyl; in particular hydrogen or acetyl or benzoyl;
for p: 1 or 2;
for R⁴: arginine, N-acetylarginine, N-t-butanoylarginine, N-benzoylarginine, N-piperonylarginine, N-glycinylarginine, N-acetylglycinylarginine, alanine, N-acetylalanine, asparagine, N-acetylasparagine, aspartic acid, N-acetylaspartic acid, lysine, N-acetyllysine, leucine, N-acetylleucine, glutamic acid, tyrosine, N-acetyltyrosine, proline or N-glycinylproline (such as arginine, N-acetylarginine, N-t-butanoylarginine, N-benzoylarginine, N-piperonylarginine, N-glycinylarginine, lysine, glutamic acid, proline and N-glycinylproline).

Generally the corresponding compound (the prodrug in accordance with the invention) is administered orally. However, in certain embodiments it is envisaged that it could be administered by another route.

Each corresponding compound may have a different release profile, the rate of release of the compound depending upon the rate at which the cleavable moiety is cleaved, and the rate in which the nitrogen nucleophile can undergo an intramolecular cyclization—release reaction thus displacing the compound. Accordingly, one embodiment of the method comprises administering a plurality of corresponding compounds to the patient, each corresponding compound having a different spacer leaving group and/or a different cleavable moiety so as to provide the patient with a different controlled release of the compound.

In a further aspect, the present invention provides a prodrug of an opioid (such as hydrocodone, oxycodone, oxymorphone or hydromorphone) that is capable of providing in vivo-activated controlled release of the opioid. Accordingly, the present invention provides a compound of general formula:

R₁R₂R₃N⁺—(C(R^{1a})(R^{2a}))—Ar—Z—C(O)—Y—(C(R¹)(R²))ₙ—N—(R³)(R⁴)A⁻ II

or a salt, hydrate or solvate thereof wherein:

R₁R₂R₃N⁺—represents a residue of an opioid wherein the lone pair of electrons of the tertiary amine nitrogen atom is replaced with a bond to —(C(R^{1a})(R^{2a})—Ar—Z—C(O)—Y—(C(R¹)(R²))ₙ—N—(R³)(R⁴);

A⁻ represents an anion; and

R^{1a}, R^{2a}, Ar, Z, Y, R¹, R², n, R³ and R⁴ have any of the meanings as defined hereinabove.

For example, when R₁R₂R₃N⁺— is a residue of hydromorphone, the compound of formula (II) has the structure:

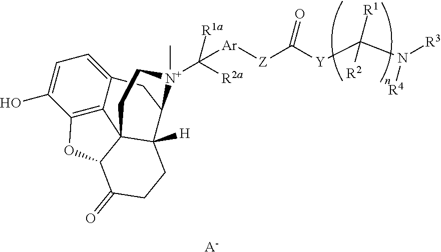

Similarly, when R₁R₂R₃N⁺— is a residue of hydrocodone, the compound of formula (II) has the structure

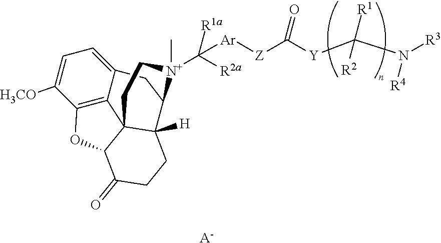

Examples of opioids include (3R,4S,beta-S)-13-fluoro ohmefentanyl, alfentanil, buprenorphine, carfentanil, codeine, diacetylmorphine, dihydrocodeine, dihydroetorphine, diprenorphine, etorphine, fentanyl, hydrocodone, hydromorphone, LAAM, levorphanol, lofentanil, meperidine, methadone, morphine, naloxone, naltrexone, beta-hydroxy 3-methylfentanyl, N-methylnaltrexone, oxycodone, oxymorphone, propoxyphene, remifentanil, sufentanil, tilidine and tramadol. Another example is N-methylnaloxone. Particular examples are hydromorphone and oxymorphone. Other particular examples are hydrocodone and oxycodone.

It will be appreciated that the quaternary nitrogen atom is chiral and accordingly the compounds of the invention may exist and be isolated in stereoisomeric forms. The present invention includes the compounds in any such form.

The anion A⁻ may conveniently be an anion derived from a pharmaceutically acceptable acid, such as an acid used to form a pharmaceutically acceptable salt.

In another aspect, pharmaceutical compositions are provided which generally comprise one or more compounds of Formula (II), salts, hydrates or solvates thereof and a pharmaceutically acceptable vehicle such as a diluent, carrier, excipient or adjuvant. The choice of diluent, carrier, excipient and adjuvant will depend upon, among other factors, the desired mode of administration.

### BRIEF DESCRIPTION OF THE DRAWINGS

FIG. 1 shows the plasma concentration time course of the production of hydrocodone following oral (PO) dosing of compounds of the present invention in rats.

### DETAILED DESCRIPTION

### Definitions

“Alkyl” by itself or as part of another substituent refers to a saturated branched or straight-chain monovalent hydrocarbon radical derived by the removal of one hydrogen atom from a single carbon atom of a parent alkane. Typical alkyl groups include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl.

In some embodiments, an alkyl group comprises from 1 to 20 carbon atoms. In other embodiments, an alkyl group comprises from 1 to 10 carbon atoms. In still other embodiments, an alkyl group comprises from 1 to 6 carbon atoms, such as from 1 to 4 carbon atoms.

“Acyl” by itself or as part of another substituent refers to a radical —C(O)R³⁰, where R³⁰ is hydrogen, alkyl, cycloalkyl, cycloheteroalkyl, aryl, arylalkyl, heteroalkyl, heteroaryl, heteroarylalkyl as defined herein. Representative examples include, but are not limited to formyl, acetyl, t-butanoyl, cyclohexylcarbonyl, cyclohexylmethylcarbonyl, benzoyl, piperonyl, benzylcarbonyl and the like.

“Alkoxy” by itself or as part of another substituent refers to a radical —OR³¹ where R³¹ represents an alkyl or cycloalkyl group as defined herein. Representative examples include, but are not limited to, methoxy, ethoxy, propoxy, butoxy, cyclohexyloxy and the like.

“Alkoxycarbonyl” by itself or as part of another substituent refers to a radical —C(O)OR³¹ where R³¹ represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

“Aryl” by itself or as part of another substituent refers to a monovalent aromatic hydrocarbon radical derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In some embodiments, an aryl group comprises from 6 to 20 carbon atoms. In other embodiments, an aryl group comprises from 6 to 12 carbon atoms. Examples of an aryl group are phenyl and naphthyl.

“Arylalkyl” by itself or as part of another substituent refers to an acyclic alkyl radical in which one of the hydrogen atoms bonded to a carbon atom, typically a terminal or sp³ carbon atom, is replaced with an aryl group. Typical arylalkyl groups include, but are not limited to, benzyl, 2-phenyleth-1-yl, naphthylmethyl, 2-naphthyleth-1-yl, naphthobenzyl, 2-naphthophenyleth-1-yl and the like. In some embodiments, an arylalkyl group is (C₇-C₃₀) arylalkyl, e.g., the alkyl moiety of the arylalkyl group is (C₁-C₁₀) and the aryl moiety is (C₆-C₂₀). In other embodiments, an arylalkyl group is (C₇-C₂₀) arylalkyl, e.g., the alkyl moiety of the arylalkyl group is (C₁-C₈) and the aryl moiety is (C₆-C₁₂).

Compounds may be identified either by their chemical structure and/or chemical name. The compounds described herein may contain one or more chiral centers and/or double bonds and therefore, may exist as stereoisomers, such as double-bond isomers (i.e., geometric isomers), enantiomers or diastereomers. Accordingly, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature.

Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms. Certain compounds may exist in multiple crystalline or amorphous forms. In general, all physical forms are equivalent for the uses contemplated herein and are intended to be within the scope of the present disclosure.

“Cycloalkyl” by itself or as part of another substituent refers to a saturated cyclic alkyl radical. Typical cycloalkyl groups include, but are not limited to, groups derived from cyclopropane, cyclobutane, cyclopentane, cyclohexane and the like. In some embodiments, the cycloalkyl group is (C₃-C₁₀) cycloalkyl. In other embodiments, the cycloalkyl group is (C₃-C₇) cycloalkyl.

“Cycloheteroalkyl” by itself or as part of another substituent, refers to a saturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Heteroalkyl” by itself or as part of another substituent refers to an alkyl group in which one or more of the carbon atoms (and any associated hydrogen atoms) are independently replaced with the same or different heteroatomic groups. Typical heteroatomic groups which can be included in these groups include, but are not limited to, —O—, —S—, —O—O—, —S—S—, —O—S—, —NR³⁷R³⁸—, ═N—N═, —N═N—N═N—NR³⁹R⁴⁰, PR⁴¹, —P(O)₂—, —POR⁴²—, —O—P(O)₂—, SO—, —SO₂—, —SnR⁴³R⁴⁴— and the like, where R³⁷, R³⁸, R³⁹, R⁴⁰, R⁴¹, R⁴², R⁴³ and R⁴⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some embodiments, the heteroaryl group is from 5-20 membered heteroaryl. In other embodiments, the heteroaryl group is from 5-10 membered heteroaryl. In still other embodiments, heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent, refers to an acyclic alkyl radical in which one of the hydrogen atoms bonded to a carbon atom, typically a terminal or sp³ carbon atom, is replaced with a heteroaryl group. In some embodiments, the heteroarylalkyl group is a 6-30 membered heteroarylalkyl, e.g., the alkyl moiety of the heteroarylalkyl is 1-10 membered and the heteroaryl moiety is a 5-20-membered heteroaryl. In other embodiments, the heteroarylalkyl group is 6-20 membered heteroarylalkyl, e.g., the alkyl moiety of the heteroarylalkyl is 1-8 membered and the heteroaryl moiety is a 5-12-membered heteroaryl.

“Parent Aromatic Ring System” by itself or as part of another substituent, refers to an unsaturated cyclic or polycyclic ring system having a conjugated it electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System” by itself or as part of another substituent, refers to a parent aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical parent heteroaromatic ring systems include, but are not limited to, arsindole, carbazole, 3-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Pharmaceutical composition” refers to at least one compound and a pharmaceutically acceptable vehicle, with which the compound is administered to a patient.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutically acceptable vehicle” refers to a diluent, adjuvant, excipient or carrier with, or in which a compound is administered.

“Patient” includes humans, but also other mammals, such as livestock, zoo animals and companion animals.

“Phenol” by itself or as part of another substituent, refers to a parent aromatic ring system in which one hydrogen atom of the parent aromatic system is replaced by a hydroxyl group.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

“Prodrug” refers to a derivative of an active agent that requires a transformation within the body to release the active agent. Prodrugs are frequently, although not necessarily, pharmacologically inactive until converted to the active agent.

“Promoiety” refers to a form of protecting group that when used to mask a functional group within an active agent converts the active agent into a prodrug. Typically, the promoiety will be attached to the drug via bond(s) that are cleaved by enzymatic or non-enzymatic means in vivo.

“Protecting group” refers to a grouping of atoms that when attached to a reactive functional group in a molecule masks, reduces or prevents reactivity of the functional group. Examples of protecting groups can be found in Green et al., “Protective Groups in Organic Chemistry,” (Wiley, 2^{nd} ed. 1991) and Harrison et al., “Compendium of Synthetic Organic Methods,” Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Substituted” refers to a group in which one or more hydrogen atoms are independently replaced with the same or different substituent(s). Typical substituents include, but are not limited to, alkylenedioxy (such as methylenedioxy), -M, —R⁶⁰, —O⁻, ═O, —OR⁶⁰, —SR⁶⁰, —S—, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O)₂O, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O, —C(S)OR⁶⁰, —NR⁶²C(O)NR⁶⁰R⁶¹, —NR⁶²C(S)NR⁶⁰R⁶¹—NR⁶²C(NR⁶³)NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In some embodiments, substituents include -M, —R⁶⁰, ═O, —OR⁶⁰, —SR⁶⁰, —S—, ═S, —NR⁶⁰R⁶¹, ═NR⁶⁰, —CF₃, —CN, —OCN, —SCN, —NO, —NO₂, ═N₂, —N₃, —S(O)₂R⁶⁰, —OS(O)₂O⁻, —OS(O)₂R⁶⁰, —P(O)(O)₂, —P(O)(OR⁶⁰)(O), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O, —NR⁶²C(O)NR⁶⁰R⁶¹. In other embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)NR⁶⁰R⁶¹, —C(O)O—. In still other embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —OP(O)(R⁶⁰)(OR⁶¹), —C(O)R⁶⁰, —C(O)OR⁶⁰, —C(O)O, where R⁶⁰, R⁶¹ and R⁶² are as defined above.

“Treating” or “treatment” of any disease or disorder refers, in some embodiments, to ameliorating the disease or disorder (i.e., arresting or reducing the development of the disease or at least one of the clinical symptoms thereof). In other embodiments “treating” or “treatment” refers to ameliorating at least one physical parameter, which may not be discernible by the patient. In yet other embodiments, “treating” or “treatment” refers to inhibiting the disease or disorder, either physically, (e.g., stabilization of a discernible symptom), physiologically, (e.g., stabilization of a physical parameter), or both. In still other embodiments, “treating” or “treatment” refers to delaying the onset of the disease or disorder.

“Therapeutically effective amount” means the amount of a compound that, when administered to a patient for treating a disease, is sufficient to effect such treatment for the disease. The “therapeutically effective amount” will vary depending on the compound, the disease and its severity and the age, weight, etc., of the patient to be treated.

Reference will now be made in detail to various embodiments. It will be understood that the invention is not limited to these embodiments. To the contrary, it is intended to cover alternatives, modifications, and equivalents as may be included within the spirit and scope of the allowed claims.

In some embodiments, Y is NR⁵ and R⁵ is hydrogen or alkyl. In other embodiments, n is 2 or 3. In other embodiments, n is 1. In still further embodiments, R¹, R², R³, R⁵ and R⁸ are independently hydrogen or alkyl.

In some embodiments, each R⁶ is independently, hydrogen, alkyl, substituted alkyl, aryl, arylalkyl, cycloalkyl, substituted cycloalkyl, substituted arylalkyl or heteroarylalkyl or optionally, R⁶ and R⁷ together with the atoms to which they are attached form a cycloheteroalkyl or substituted cycloheteroalkyl ring. In other embodiments, R⁶ is independently hydrogen, alkyl, substituted alkyl, aryl, arylalkyl, substituted arylalkyl, heteroalkyl, heteroarylalkyl, substituted heteroarylalkyl, or optionally, R⁶ and R⁷ together with the atoms to which they are bonded form a cycloheteroalkyl or substituted cycloheteroalkyl ring. In still other embodiments, each R⁶ is independently, hydrogen, methyl, isopropyl, isobutyl, sec-butyl, t-butyl, cyclopentyl, cyclohexyl, —CH₂OH, —CH(OH)CH₃, —CH₂CO₂H, —CH₂CH₂CO₂H, —CH₂CONH₂, —CH₂CH₂CONH₂, —CH₂CH₂SCH₃, —CH₂SH, —CH₂ (CH₂)₃NH₂, —CH₂CH₂CH₂NHC(NH)NH₂, phenyl, benzyl, homobenzyl, 4-hydroxybenzyl, 4-bromobenzyl, 4-imidazolylmethyl, 3-indolylmethyl, 3-[5-hydroxyindolyl]-methyl, 9-anthranylmethyl, 3-benzothienylmethyl, cyclohexylmethyl, diphenylmethyl, 2-furylmethyl, iodomethyl, 1-napthylmethyl, 2-napthylmethyl, 2-pyridylmethyl, 3-pyridylmethyl, 4-pyridylmethyl, 3-styrylmethyl, 2-thienylmethyl, vinylmethyl, cyclohexyl, acetylenomethyl, 2-trifluoromethylbenzyl, 2-chlorobenzyl, 2-cyanobenzyl, 2-fluorobenzyl, 2-methylbenzyl, 3-trifluoromethylbenzyl, 3-chlorobenzyl, 3-cyanobenzyl, 3-fluorobenzyl, 3-methylbenzyl, 4-benzoylbenzyl, 3,5-dibromo-4-hydroxybenzyl, 3-trifluoromethylbenzyl, 4-chlorobenzyl, 4-cyanobenzyl, 4-fluorobenzyl, 4-iodobenzyl, 4-methylbenzyl, 4-nitrobenzyl, 3,4-dihydroxybenzyl, 2,4-dichlorobenzyl, 3,4 dichlorobenzyl, 3,4 difluorobenzyl, 3,5 diiodo-4-hydroxylbenzyl, 3-nitro-4-hydroxybenzyl, aminomethyl,

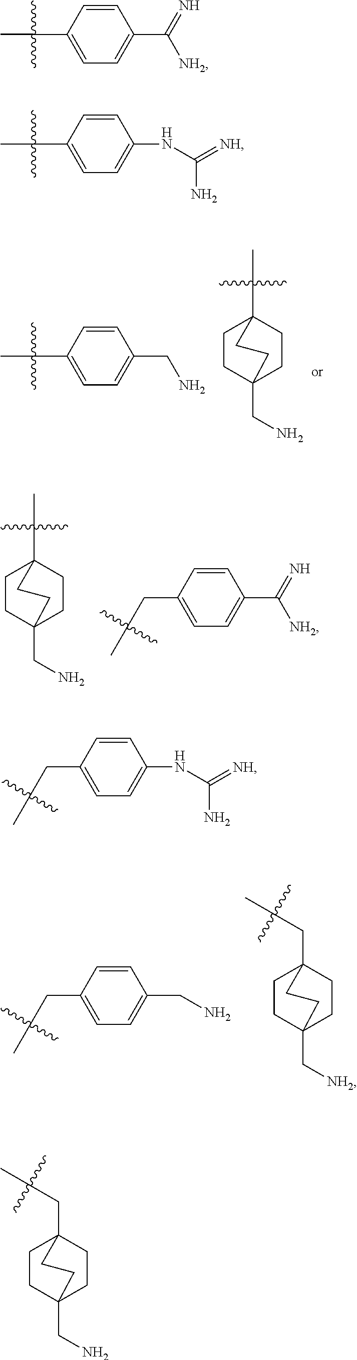

or optionally R⁶ and R⁷ together with the atoms to which they are attached form an azetidine, pyrrolidine or piperidine ring.

In some embodiments, W is —NR⁸ and each R⁷ is independently hydrogen or alkyl, aryl or arylalkyl.

In some embodiments, R⁷ is hydrogen, alkyl, acyl or alkoxycarbonyl.

In other embodiments, each R⁶ is independently —CH₂ (CH₂)₃NH₂ or —CH₂CH₂CH₂NHC(NH)NH₂. In still other embodiments, p is 1 and R⁶ is —CH₂ (CH₂)₃NH₂ or —CH₂CH₂CH₂NHC(NH)NH₂. In still other embodiments, each W is —NR⁸—, each R⁸ is hydrogen and R⁷ is hydrogen, acyl, substituted acyl, alkoxycarbonyl or substituted alkoxycarbonyl.

In some embodiments, each R⁶ is independently phenyl, benzyl, 4-hydroxybenzyl, 4-bromobenzyl, 4-imidazolylmethyl, 3-indolylmethyl, isobutyl, —CH₂CH₂SCH₃, —CH₂CH₂CONH₂, —CH₂CH₂CONH₂ or —CH₂CO₂H. In still other embodiments, each R⁶ is independently benzyl, 4-hydroxybenzyl, 4-bromobenzyl or 3-indolylmethyl. In still other embodiments, n is 1 and R⁶ is phenyl, benzyl, 4-hydroxybenzyl, 4-bromobenzyl, 4-imidazolylmethyl, 3-indolylmethyl, isobutyl, —CH₂CH₂SCH₃, —CH₂CH₂CONH₂, —CH₂CH₂CONH₂ or —CH₂CO₂H. In still other embodiments, n is 1 and R⁶ is benzyl, 4-hydroxybenzyl, 4-bromobenzyl or 3-indolylmethyl. In some of any of the above embodiments, each W is —NR⁸—, each R⁸ is hydrogen and R⁷ is acyl, substituted acyl, alkoxycarbonyl or substituted alkoxycarbonyl.

In some embodiments, p is greater than 1 and R⁷ is hydrogen. In any of the above embodiments, each W is —NR^{s}—, each R⁸ is hydrogen and R⁷ is acyl, substituted acyl, alkoxycarbonyl or substituted alkoxycarbonyl.

In some embodiments, p is 3 and R⁷ is hydrogen. In other embodiments, each W is —NR⁸— and each R⁸ is hydrogen.

In some embodiments, each R⁶ is independently hydrogen, methyl, isopropyl, isobutyl, sec-butyl, —CH₂OH or —CH₂SH. In other embodiments, p is 1 and R⁶ is hydrogen, methyl, isopropyl, isobutyl or sec-butyl, each W is —NR⁸—, each R⁸ is hydrogen and R⁷ is acyl, substituted acyl, alkoxycarbonyl or substituted alkoxycarbonyl.

In some embodiments, each R⁶ is independently hydrogen, methyl, isopropyl, isobutyl, sec-butyl, t-butyl, cyclopentyl, cyclohexyl, —CH₂OH, —CH(OH)CH₃, —CH₂CONH₂, —CH₂CH₂SCH₃, —CH₂SH, phenyl, benzyl, 4-hydroxybenzyl, 4-bromobenzyl or 3-indolylmethyl. In other embodiments, each R⁶ is independently hydrogen, methyl, isopropyl, isobutyl, sec-butyl, t-butyl, cyclopentyl, cyclohexyl, phenyl, benzyl, 4-bromobenzyl, 3-indolylmethyl or optionally R⁶ and R⁷ together with the atoms to which they are attached form an azetidine, pyrrolidine or piperidine ring. In some of the above embodiments, each W is —NR⁸—, each R⁸ is hydrogen or optionally each R⁶ and R⁸, independently together with the atoms to which they are attached form an azetidine, pyrrolidine or piperidine ring and R⁷ is acyl, substituted acyl, alkoxycarbonyl or substituted alkoxycarbonyl.

In some embodiments, each R⁶ is independently benzyl, 4-hydroxybenzyl or isobutyl.

In other embodiments, each W is —NR⁸—, each R⁸ is hydrogen and R⁷ is acyl, substituted acyl, alkoxycarbonyl or substituted alkoxycarbonyl.

In some embodiments, each R⁶ is independently —CH₂CO₂H or —CH₂CH₂CO₂H. In other embodiments, each W is —NR⁸—, each R⁸ is hydrogen and R⁷ is acyl, substituted acyl, alkoxycarbonyl or substituted alkoxycarbonyl.

In some embodiments, p is 2 and the R⁶ group adjacent to the N-terminal nitrogen atom is independently, hydrogen, methyl, isopropyl, isobutyl, sec-butyl, t-butyl, cyclopentyl, cyclohexyl, —CH₂OH, —CH(OH)CH₃, —CH₂CO₂H, —CH₂CH₂CO₂H, —CH₂CONH₂, —CH₂CH₂CONH₂, —CH₂CH₂SCH₃, —CH₂SH, —CH₂ (CH₂)₃NH₂, —CH₂CH₂CH₂NHC(NH)NH₂, phenyl, benzyl, homobenzyl (phenethyl), 4-hydroxybenzyl, 4-bromobenzyl, 4-imidazolylmethyl, 3-indolylmethyl, 3-[5-hydroxyindolyl]-methyl, 9-anthranylmethyl, 3-benzothienylmethyl, cyclohexylmethyl, diphenylmethyl, 2-furylmethyl, iodomethyl, 1-napthylmethyl, 2-napthylmethyl, 2-pyridylmethyl, 3-pyridylmethyl, 4-pyridylmethyl, 3-styrylmethyl, 2-thienylmethyl, vinylmethyl, cyclohexyl, acetylenomethyl, 2-trifluoromethylbenzyl, 2-chlorobenzyl, 2-cyanobenzyl, 2-fluorobenzyl, 2-methylbenzyl, 3-trifluoromethylbenzyl, 3-chlorobenzyl, 3-cyanobenzyl, 3-fluorobenzyl, 3-methylbenzyl, 4-benzoylbenzyl, 3,5-dibromo-4-hydroxybenzyl, 3-trifluoromethylbenzyl, 4-chlorobenzyl, 4-cyanobenzyl, 4-fluorobenzyl, 4-iodobenzyl, 4-methylbenzyl, 4-nitrobenzyl, 3,4-dihydroxybenzyl, 2,4-dichlorobenzyl, 3,4 dichlorobenzyl, 3,4 difluorobenzyl, 3,5 diiodo-4-hydroxylbenzyl, 3-nitro-4-hydroxybenzyl, aminomethyl,

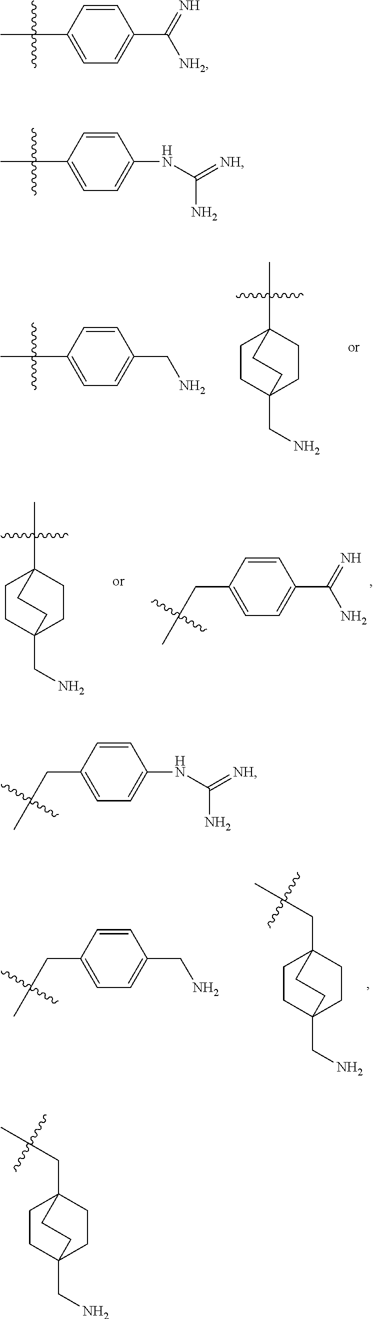

or optionally each R⁶ and R⁸, independently together with the atoms to which they are attached form an azetidine, pyrrolidine or piperidine ring and the other R⁶ group is methyl or R⁶ and R⁸, independently together with the atoms to which they are attached form a pyrrolidine ring. In other embodiments, each W is —NR⁸—, each R⁸ is hydrogen or optionally each R⁶ and R⁸, independently together with the atoms to which they are attached form a pyrrolidine ring and R⁷ is acyl, substituted acyl, alkoxycarbonyl or substituted alkoxycarbonyl.

In some of the above embodiments, p is 1, and R⁶ is hydrogen. In some of the above embodiments, p is 1, R⁶ is hydrogen and W is NH. In some of the above embodiments, p is 1, p⁶ is hydrogen, W is NH and R⁷ is hydrogen. In other embodiments, each R⁶ is hydrogen and W is NH. In still other embodiments, each R⁶ is hydrogen, W is NH and R⁷ is hydrogen.

In some embodiments, Y is NR⁵, n is 2 or 3, p is 1 or 2, R¹, R², R³, R⁵ and R⁷ are independently hydrogen or alkyl, each R⁶ is independently hydrogen, alkyl, substituted alkyl, aryl, arylalkyl, substituted arylalkyl, heteroalkyl, heteroarylalkyl, substituted heteroarylalkyl or optionally, R⁶ and R⁷ together with the atoms to which they are bonded form a cycloheteroalkyl or substituted cycloheteroalkyl ring. In other embodiments, Y is NR⁵, n is 2, p is 1, R¹ and R² are hydrogen, R³ and R⁵ are methyl or hydrogen and R⁶ is independently hydrogen, alkyl, substituted alkyl, aryl, arylalkyl, substituted arylalkyl, heteroalkyl, heteroarylalkyl, substituted heteroarylalkyl or optionally, R⁶ and R⁷ together with the atoms to which they are bonded form a cycloheteroalkyl or substituted cycloheteroalkyl ring or optionally R⁷ is hydrogen. In still other embodiments, Y is NR⁵, n is 2, R¹ and R² are hydrogen, R³ and R⁵ are methyl or hydrogen, R⁷ is hydrogen and R⁶ is —CH₂ (CH₂)₃NH₂ or —CH₂CH₂CH₂NHC(NH)NH₂.

### Methods of Synthesis

The compounds described herein may be obtained via the routes generically illustrated in Schemes 1-4.

The promoieties described herein, may be prepared and attached to compounds containing phenols by procedures known to those of skill in the art (See e.g., Green et al., “Protective Groups in Organic Chemistry,” (Wiley, 2^{nd} ed. 1991); Harrison et al., “Compendium of Synthetic Organic Methods,” Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-17, (Wiley Interscience); Trost et al., “Comprehensive Organic Synthesis,” (Pergamon Press, 1991); “Theilheimer's Synthetic Methods of Organic Chemistry,” Volumes 1-45, (Karger, 1991); March, “Advanced Organic Chemistry,” (Wiley Interscience), 1991; Larock “Comprehensive Organic Transformations,” (VCH Publishers, 1989); Paquette, “Encyclopedia of Reagents for Organic Synthesis,” (John Wiley & Sons, 1995), Bodanzsky, “Principles of Peptide Synthesis,” (Springer Verlag, 1984); Bodanzsky, “Practice of Peptide Synthesis,” (Springer Verlag, 1984). Further, starting materials may be obtained from commercial sources or via well established synthetic procedures, supra.

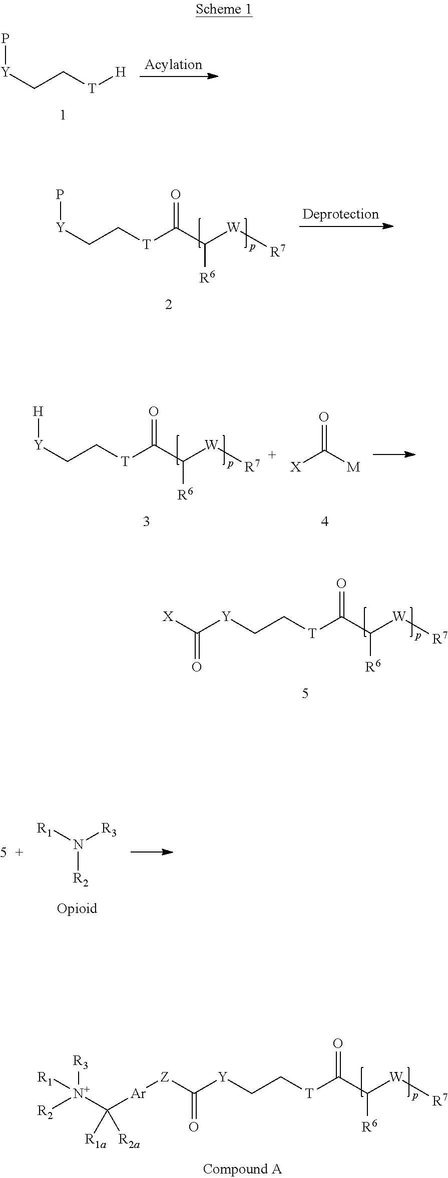

Referring now to Scheme 1 and formula II, supra, where for illustrative purposes T is —NR³, Y is NR⁵, —O— or —S—, W is NR⁸, —O— or —S—, n is 2, R¹ and R² are hydrogen, p, R³, R⁵, R⁶, R⁷ and R⁸ are as previously defined, R₁R₂R₃N— represents a residue of an opioid, X is an appropriate optionally substituted phenol, optionally substituted thiol, or optionally substituted aniline (e.g. a compound of formula HO—(C(R^{1a})(R^{2a}))—Ar—ZH), P is a protecting group, and M is a leaving group, compound 1 may be acylated with an appropriate carboxylic acid or carboxylic acid equivalent to provide compound 2 which then may be deprotected to yield compound 3. Compound 3 is then reacted with an activated carbonic acid equivalent 4 to provide desired compound 5. Compound 5 is then coupled to the tertiary nitrogen of an opioid to complete the synthesis of Compound A.

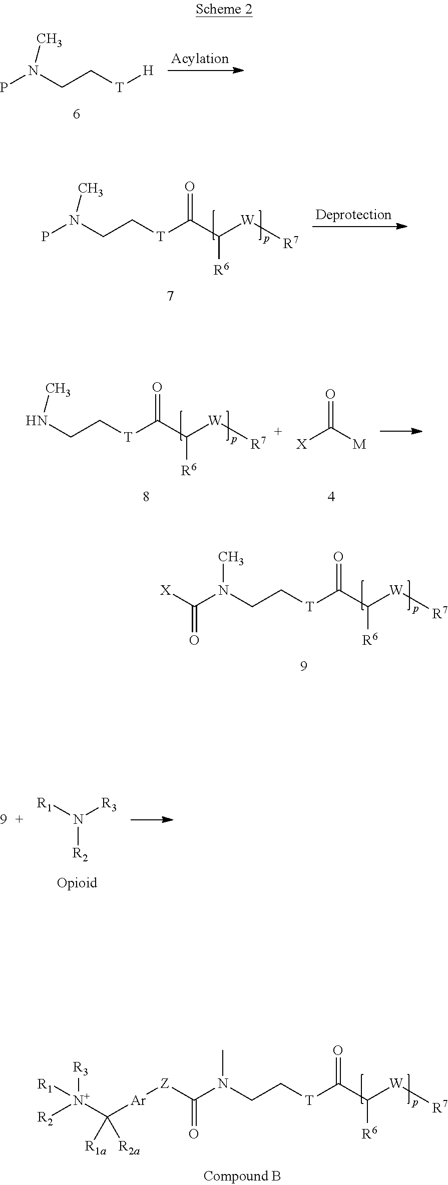

Referring now to Scheme 2 and formula II, supra, where for illustrative purposes T is —NR³, Y is NCH₃, W is NR⁸, —O— or —S—, n is 2, R¹ and R² are hydrogen, p, R³, R⁶, R⁷ and R⁸ are as previously defined, R₁R²R₃N— represents a residue of an opioid, X is an appropriate optionally substituted phenol, optionally substituted thiol, or optionally substituted aniline, P is a protecting group, and M is a leaving group, compound 6 is acylated with an appropriate carboxylic acid or carboxylic acid equivalent to provide compound 7. Compound 7 is then deprotected and reacted with activated carbonic acid equivalent 4 to provide desired compound 9. Compound 9 is then coupled to the tertiary nitrogen of an opioid to complete the synthesis of Compound B.

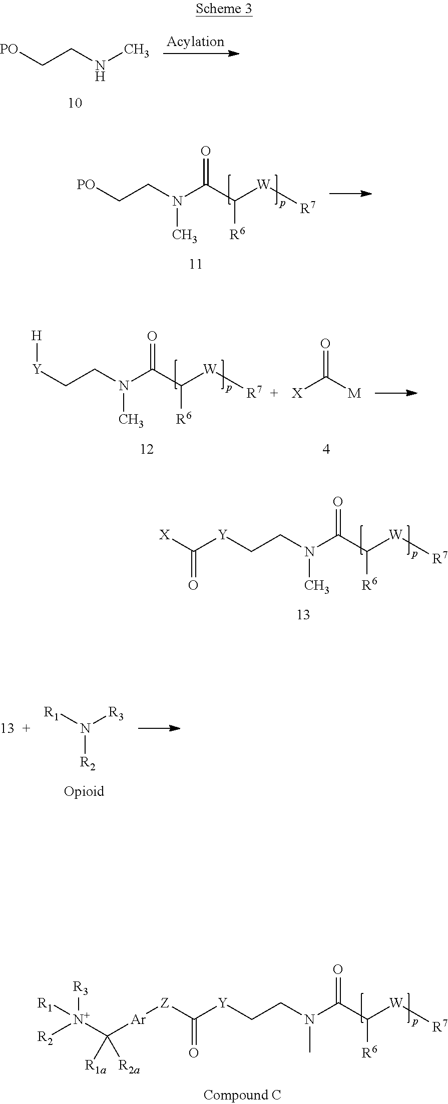

Referring now to Scheme 3 and formula II, supra, where for illustrative purposes T is NCH₃, Y is NR⁵, —O— or —S—, W is NR⁸, —O— or —S—, n is 2, R¹ and R² are hydrogen, p, R⁵, R⁶, R⁷ and R⁸ are as previously defined, R₁R₂R₃N— represents a residue of an opioid, X is an appropriate optionally substituted phenol, optionally substituted thiol, or optionally substituted aniline, P is a protecting group, and M is a leaving group, compound 10 is acylated with an appropriate carboxylic acid or carboxylic acid equivalent to provide compound 11 which after deprotection and functional group intraconversion, if necessary, is converted to compound 12. Reaction of compound 12 with activated carbonic acid equivalent 4 provides desired compound 13. Compound 13 is then coupled to the tertiary nitrogen of an opioid to complete the synthesis of Compound C.

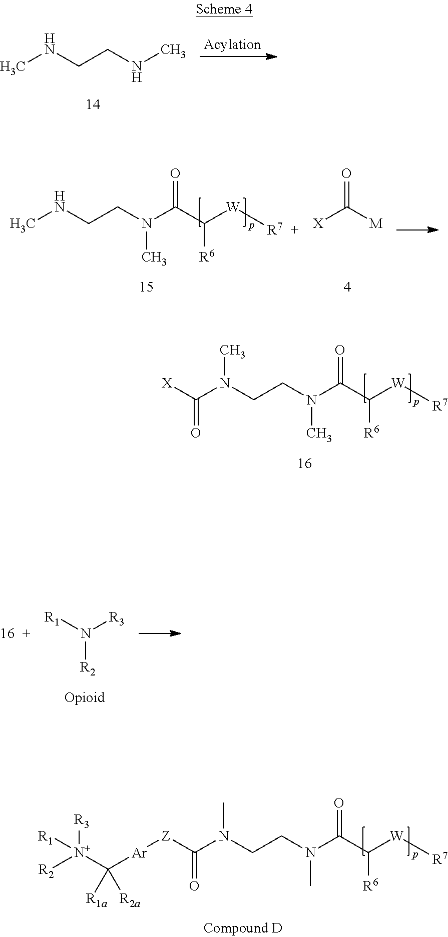

Referring now to Scheme 4 and formula II, supra, where for illustrative purposes T and Y are NCH₃, W is NR⁸, —O— or —S—, n is 2, R¹ and R² are hydrogen, p, R⁶, R⁷ and R⁸ are as previously defined, R₁R₂R₃N— represents a residue of an opioid, X is an appropriate optionally substituted phenol, optionally substituted thiol, or optionally substituted aniline, P is a protecting group, and M is a leaving group, compound 14 is acylated with an appropriate carboxylic acid or carboxylic acid equivalent to provide compound 15. Reaction of compound 15 with activated carbonic acid equivalent 4 provides desired compound 16. Compound 16 is then coupled to the tertiary nitrogen of an opioid to complete the synthesis of Compound D.

Selection of appropriate protecting groups, reagents and reaction conditions for any of the steps in the above Schemes is well within the ambit of those of skilled in the art. Other methods for synthesis of the prodrugs described herein will be readily apparent to the skilled artisan and may be used to synthesize the compounds described herein. Accordingly, the methods presented in the Schemes herein are illustrative rather than comprehensive.

Compounds of formula (II) may be prepared following the method described herein, by forming a compound in which X represents, for example, a p-hydroxymethylaryloxy group, converting this compound into a corresponding compound in which the hydroxy group is replaced with a leaving atom or group, and then using this compound as a reagent to alkylate the amine group of an opioid, such as hydrocodone, oxycodone, hydromorphone or oxymorphone. Thereafter, if desired, a resultant compound of formula (II) in which R⁷ represents hydrogen may be acylated, for example to increase p or to afford a compound in which R⁷ is an acyl group.

According to another aspect, therefore, the present invention provides a process for the preparation of a compound of formula (II) or a pharmaceutically acceptable salt thereof, which comprises reacting a compound of formula (V)

M¹(C(R^{1a})(R^{2a}))—Ar—Z—C(O)—Y—(C(R¹)(R²))ₙ—N(R³)(R⁴) (V)

or a protected derivative thereof, in which M¹ represents a leaving atom or group, such as a chlorine atom, with an opioid (such as hydrocodone, oxycodone, hydromorphone or oxymorphone), followed by removing any protecting groups and, if desired, acylating a compound of formula (II) in which R⁷ (in the group R⁴ as defined hereinabove) represents a hydrogen atom and/or forming a pharmaceutically acceptable salt.

The reaction is conveniently performed in the presence of a lithium salt, such as lithium bromide. Convenient solvents include amides, such as dimethylformamide.

As described above, the intermediates of formula (V) can be prepared from the corresponding alcohol of formula (VI)

HO—(C(R^{1a})(R^{2a}))—Ar—Z—C(O)—Y—(C(R¹)(R²))ₙ—N—(R³)(R⁴) (VI)

or a protected derivative thereof, for example by reaction with thionyl chloride to afford the chloride.

The invention further provides all the novel intermediates described herein.

### Therapeutic Methods of Use

In general, the prodrugs disclosed herein may be used to treat and/or prevent the same disease(s) and/or conditions as the parent drug which are well known in the art (see, e.g., Physicians Desk Reference, 2000 54^{th} Edition and the Merck Index, 13^{th} Edition).

For example, a prodrug of hydroxycodone, oxymorphone, or hydromorphone could be used, inter alia, to treat or prevent pain including, but not limited to include, acute pain, chronic pain, neuropathic pain, acute traumatic pain, arthritic pain, osteoarthritic pain, rheumatoid arthritic pain, muscular skeletal pain, post-dental surgical pain, dental pain, myofascial pain, cancer pain, visceral pain, diabetic pain, muscular pain, post-herpetic neuralgic pain, chronic pelvic pain, endometriosis pain, pelvic inflammatory pain and child birth related pain. Acute pain includes, but is not limited to, acute traumatic pain or post-surgical pain. Chronic pain includes, but is not limited to, neuropathic pain, arthritic pain, osteoarthritic pain, rheumatoid arthritic pain, muscular skeletal pain, dental pain, myofascial pain, cancer pain, diabetic pain, visceral pain, muscular pain, post-herpetic neuralgic pain, chronic pelvic pain, endometriosis pain, pelvic inflammatory pain and back pain.

### Pharmaceutical Compositions

The pharmaceutical compositions disclosed herein comprise a prodrug disclosed herein with a suitable amount of a pharmaceutically acceptable vehicle, so as to provide a form for proper administration to a subject.

Suitable pharmaceutical vehicles include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions may be manufactured by means of conventional mixing, dissolving, granulating, dragee-making, levigating, emulsifying, encapsulating, entrapping or lyophilizing processes. Pharmaceutical compositions may be formulated in a conventional manner using one or more physiologically acceptable carriers, diluents, excipients or auxiliaries, which facilitate processing of compositions and compounds disclosed herein into preparations which can be used pharmaceutically. Proper formulation is dependent upon the route of administration chosen.

The present pharmaceutical compositions can take the form of solutions, suspensions, emulsion, tablets, pills, pellets, capsules, capsules containing liquids, powders, sustained-release formulations, suppositories, emulsions, aerosols, sprays, suspensions or any other form suitable for use known to the skilled artisan. In some embodiments, the pharmaceutically acceptable vehicle is a capsule (see e.g., Grosswald et al., U.S. Pat. No. 5,698,155). Other examples of suitable pharmaceutical vehicles have been described in the art (see Remington's Pharmaceutical Sciences, Philadelphia College of Pharmacy and Science, 19th Edition, 1995).

Pharmaceutical compositions for oral delivery may be in the form of tablets, lozenges, aqueous or oily suspensions, granules, powders, emulsions, capsules, syrups, slurries, suspensions or elixirs, for example. Orally administered compositions may contain one or more optional agents, for example, sweetening agents such as fructose, aspartame or saccharin, flavoring agents such as peppermint, oil of wintergreen, or cherry coloring agents and preserving agents, to provide a pharmaceutically palatable preparation. Moreover, when in tablet or pill form, the compositions may be coated to delay disintegration and absorption in the gastrointestinal tract, thereby providing a sustained action over an extended period of time. Oral compositions can include standard vehicles such as mannitol, lactose, starch, magnesium stearate, sodium saccharine, cellulose, magnesium carbonate, sucrose, sorbitol, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP), granulating agents, binding agents and disintegrating agents such as the cross-linked polyvinylpyrrolidone, agar, or alginic acid or a salt thereof such as sodium alginate etc.

In some embodiments, pharmaceutical compositions are in the form of lozenges or lollipops where dissolution and release of the active ingredients occurs in the oral cavity, generally through the oral mucosa. For these embodiments, buffering agents may also be used to provide an optimum environment for delivery of the agents or compositions. Additional components may include, for example, sweeteners, binders, diluents, disintegrating agents, lubricating agents, etc.

In still other embodiments, the pharmaceutical composition is a dissolving sublingual tablet, where dissolution and release of the active ingredients occurs under the tongue, and the compositions and/or compounds disclosed herein are absorbed through the oral mucosa. In these embodiments, buffering agents may also be used to provide an optimum environment for delivery of each of the agents. Additional components may include, for example, sweeteners, binders, diluents, disintegrating agents, etc.

The methods that involve oral administration of compounds disclosed herein of can also be practiced with a number of different dosage forms, which provide sustained release.

In some embodiments, the dosage form is comprised of beads that on dissolution or diffusion release compositions and/or compounds disclosed herein over an extended period of hours, preferably, over a period of at least 6 hours, more preferably, over a period of at least 8 hours and even more preferably, over a period of at least 12 hours and most preferably, over a period of at least 24 hours. The beads may have a central composition or core comprising compounds disclosed herein and pharmaceutically acceptable vehicles, including optional lubricants, antioxidants and buffers. The beads may be medical preparations with a diameter of about 1 to about 2 mm. Individual beads may comprise doses of the compounds disclosed herein. The beads, in some embodiments, are formed of non-cross-linked materials to enhance their discharge from the gastrointestinal tract. The beads may be coated with a release rate-controlling polymer that gives a timed-release profile.

The time-release beads may be manufactured into a tablet for therapeutically effective administration. The beads can be made into matrix tablets by direct compression of a plurality of beads coated with, for example, an acrylic resin and blended with excipients such as hydroxypropylmethyl cellulose. The manufacture of beads has been disclosed in the art (Lu, Int. J. Pharm. 1994, 112, 117-124; Pharmaceutical Sciences by Remington, 14^{th} ed, pp 1626-1628 (1970); Fincher, J. Pharm. Sci. 1968, 57, 1825-1835; Benedikt, U.S. Pat. No. 4,083,949) as has the manufacture of tablets (Pharmaceutical Sciences, by Remington, 17^{th} Ed, Ch. 90, pp 1603-1625 (1985).

In other embodiments, an oral sustained release pump may be used (Langer, supra; Sefton, 1987, CRC Crit. RefBiomed. Eng. 14:201; Saudek et al., 1989, N. Engl. J. Med. 321:574).

In still other embodiments, polymeric materials can be used (See “Medical Applications of Controlled Release,” Langer and Wise (eds.), CRC Press., Boca Raton, Fla. (1974); “Controlled Drug Bioavailability,” Drug Product Design and Performance, Smolen and Ball (eds.), Wiley, New York (1984); Langer et al., 1983, J Macromol. Sci. Rev. Macromol Chem. 23:61; Levy et al., 1985, Science 228: 190; During et al., 1989, Ann. Neurol. 25:351; Howard et al., 1989, J. Neurosurg. 71:105). In some embodiments, polymeric materials are used for oral sustained release delivery. Such polymers include, for example, sodium carboxymethylcellulose, hydroxypropylcellulose, hydroxypropylmethylcellulose and hydroxyethylcellulose (most preferred, hydroxypropylmethylcellulose). Other cellulose ethers have been described (Alderman, Int. J. Pharm. Tech. & Prod. Mfr. 1984, 5(3)1-9). Factors affecting drug release are well known to the skilled artisan and have been described in the art (Bamba et al., Int. J. Pharm. 1979, 2, 307).

In still other embodiments, enteric-coated preparations can be used for oral sustained release administration. Coating materials include, for example, polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In yet other embodiments, drug-releasing lipid matrices can be used for oral sustained release administration. For example, solid microparticles of compositions and/or compounds disclosed herein may be coated with a thin controlled release layer of a lipid (e.g., glyceryl behenate and/or glyceryl palmitostearate) as disclosed in Farah et al., U.S. Pat. No. 6,375,987 and Joachim et al., U.S. Pat. No. 6,379,700. The lipid-coated particles can optionally be compressed to form a tablet. Another controlled release lipid-based matrix material which is suitable for sustained release oral administration comprises polyglycolized glycerides as disclosed in Roussin et al., U.S. Pat. No. 6,171,615.

In yet other embodiments, waxes can be used for oral sustained release administration.

Examples of suitable sustained releasing waxes are disclosed in Cain et al., U.S. Pat. No. 3,402,240 (carnauba wax, candedilla wax, esparto wax and ouricury wax); Shtohryn et al., U.S. Pat. No. 4,820,523 (hydrogenated vegetable oil, bees wax, caranuba wax, paraffin, candelillia, ozokerite and mixtures thereof); and Walters, U.S. Pat. No. 4,421,736 (mixture of paraffin and castor wax).

In still other embodiments, osmotic delivery systems are used for oral sustained release administration (Verma et al., Drug Dev. Ind. Pharm. 2000, 26:695-708). In some embodiments, OROS® systems made by Alza Corporation, Mountain View, Calif. are used for oral sustained release delivery devices (Theeuwes et al., U.S. Pat. No. 3,845,770; Theeuwes et al., U.S. Pat. No. 3,916,899).

In yet other embodiments, a controlled-release system can be placed in proximity of the target of the compositions and/or compounds disclosed herein thus requiring only a fraction of the systemic dose (See, e.g., Goodson, in “Medical Applications of Controlled Release,” supra, vol. 2, pp. 115-138 (1984)). Other controlled-release systems are discussed in Langer, 1990, Science 249:1527-1533 may also be used.

In still other embodiments, the dosage form comprises compounds disclosed herein coated on a polymer substrate. The polymer can be an erodible or a nonerodible polymer. The coated substrate may be folded onto itself to provide a bilayer polymer drug dosage form. For example, compounds disclosed herein can be coated onto a polymer such as a polypeptide, collagen, gelatin, polyvinyl alcohol, polyorthoester, polyacetyl, or a polyorthocarbonate and the coated polymer folded onto itself to provide a bilaminated dosage form. In operation, the bioerodible dosage form erodes at a controlled rate to dispense the compounds over a sustained release period. Representative biodegradable polymers comprise a member selected from the group consisting of biodegradable poly(amides), poly (amino acids), poly(esters), poly(lactic acid), poly(glycolic acid), poly(carbohydrate), poly(orthoester), poly (orthocarbonate), poly(acetyl), poly(anhydrides), biodegradable poly(dihydropyrans), and poly(dioxinones) which are known in the art (Rosoff, Controlled Release of Drugs, Chap. 2, pp. 53-95 (1989); Heller et al., U.S. Pat. No. 3,811,444; Michaels, U.S. Pat. No. 3,962,414; Capozza, U.S. Pat. No. 4,066,747; Schmitt, U.S. Pat. No. 4,070,347; Choi et al., U.S. Pat. No. 4,079,038; Choi et al., U.S. Pat. No. 4,093,709).

In other embodiments, the dosage form comprises compounds disclosed herein loaded into a polymer that releases the drug(s) by diffusion through a polymer, or by flux through pores or by rupture of a polymer matrix. The drug delivery polymeric dosage form comprises a concentration of 10 mg to 2500 mg homogenously contained in or on a polymer. The dosage form comprises at least one exposed surface at the beginning of dose delivery. The non-exposed surface, when present, is coated with a pharmaceutically acceptable material impermeable to the passage of the drug(s). The dosage form may be manufactured by procedures known in the art. An example of providing a dosage form comprises blending a pharmaceutically acceptable carrier like polyethylene glycol, with a known dose of compositions and/or compounds disclosed herein at an elevated temperature, (e.g., 37° C.), and adding it to a silastic medical grade elastomer with a cross-linking agent, for example, octanoate, followed by casting in a mold. The step is repeated for each optional successive layer. The system is allowed to set for about 1 hour, to provide the dosage form.

Representative polymers for manufacturing the dosage form comprise a member selected from the group consisting of olefin, and vinyl polymers, addition polymers, condensation polymers, carbohydrate polymers, and silicone polymers as represented by polyethylene, polypropylene, polyvinyl acetate, polymethylacrylate, polyisobutylmethacrylate, poly alginate, polyamide and polysilicone. The polymers and procedures for manufacturing them have been described in the art (Coleman et al., Polymers 1990, 31, 1187-1231; Roerdink et al., Drug Carrier Systems 1989, 9, 57-10; Leong et al., Adv. Drug Delivery Rev. 1987, 1, 199-233; Roff et al., Handbook of Common Polymers 1971, CRC Press; Chien et al., U.S. Pat. No. 3,992,518).

In other embodiments, the dosage form comprises a plurality of tiny pills. The tiny time-release pills provide a number of individual doses for providing various time doses for achieving a sustained-release drug delivery profile over an extended period of time up to 24 hours. The matrix comprises a hydrophilic polymer selected from the group consisting of a polysaccharide, agar, agarose, natural gum, alkali alginate including sodium alginate, carrageenan, fucoidan, furcellaran, laminaran, hypnea, gum arabic, gum ghatti, gum karaya, gum tragacanth, locust bean gum, pectin, amylopectin, gelatin, and a hydrophilic colloid. The hydrophilic matrix comprises a plurality of 4 to 50 tiny pills, each tiny pill comprises a dose population of from 10 ng, 0.5 mg, 1 mg, 1.2 mg, 1.4 mg, 1.6 mg, 5.0 mg, etc. The tiny pills comprise a release rate-controlling wall of 0.001 mm up to 10 mm thickness to provide for the timed release of drug(s). Representative wall forming materials include a triglyceryl ester selected from the group consisting of glyceryl tristearate, glyceryl monostearate, glyceryl dipalmitate, glyceryl laureate, glyceryl didecenoate and glyceryl tridenoate. Other wall forming materials comprise polyvinyl acetate, phthalate, methylcellulose phthalate and microporous olefins. Procedures for manufacturing tiny pills are disclosed in Urquhart et al., U.S. Pat. No. 4,434,153; Urquhart et al., U.S. Pat. No. 4,721,613; Theeuwes, U.S. Pat. No. 4,853,229; Barry, U.S. Pat. No. 2,996,431; Neville, U.S. Pat. No. 3,139,383; Mehta, U.S. Pat. No. 4,752,470.

In other embodiments, the dosage form comprises an osmotic dosage form, which comprises a semipermeable wall that surrounds a therapeutic composition comprising compounds disclosed herein. In use within a subject, the osmotic dosage form comprising a homogenous composition, imbibes fluid through the semipermeable wall into the dosage form in response to the concentration gradient across the semipermeable wall. The therapeutic composition in the dosage form develops osmotic pressure differential that causes the therapeutic composition to be administered through an exit from the dosage form over a prolonged period of time up to 24 hours (or even in some cases up to 30 hours) to provide controlled and sustained release. These delivery platforms can provide an essentially zero order delivery profile as opposed to the spiked profiles of immediate release formulations.

In other embodiments, the dosage form comprises another osmotic dosage form comprising a wall surrounding a compartment, the wall comprising a semipermeable polymeric composition permeable to the passage of fluid and substantially impermeable to the passage of compounds disclosed herein present in the compartment, a drug-containing layer composition in the compartment, a hydrogel push layer composition in the compartment comprising an osmotic formulation for imbibing and absorbing fluid for expanding in size for pushing the drug composition layer from the dosage form, and at least one passageway in the wall for releasing the composition. The method delivers compounds disclosed herein by imbibing fluid through the semipermeable wall at a fluid imbibing rate determined by the permeability of the semipermeable wall and the osmotic pressure across the semipermeable wall causing the push layer to expand, thereby delivering the compounds disclosed herein from the dosage form through the exit passageway to a subject over a prolonged period of time (up to 24 or even 30 hours). The hydrogel layer composition may comprise 10 mg to 1000 mg of a hydrogel such as a member selected from the group consisting of a polyalkylene oxide of 1,000,000 to 8,000,000 weight-average molecular weight which are selected from the group consisting of a polyethylene oxide of 1,000,000 weight-average molecular weight, a polyethylene oxide of 2,000,000 molecular weight, a polyethylene oxide of 4,000,000 molecular weight, a polyethylene oxide of 5,000,000 molecular weight, a polyethylene oxide of 7,000,000 molecular weight and a polypropylene oxide of the 1,000,000 to 8,000,000 weight-average molecular weight; or 10 mg to 1000 mg of an alkali carboxymethylcellulose of 10,000 to 6,000,000 weight average molecular weight, such as sodium carboxymethylcellulose or potassium carboxymethylcellulose. The hydrogel expansion layer comprises 0.0 mg to 350 mg, in present manufacture; 0.1 mg to 250 mg of a hydroxyalkylcellulose of 7,500 to 4,500,00 weight-average molecular weight (e.g., hydroxymethylcellulose, hydroxyethylcellulose, hydroxypropylcellulose, hydroxybutylcellulose or hydroxypentylcellulose) in present manufacture; 1 mg to 50 mg of an osmagent selected from the group consisting of sodium chloride, potassium chloride, potassium acid phosphate, tartaric acid, citric acid, raffinose, magnesium sulfate, magnesium chloride, urea, inositol, sucrose, glucose and sorbitol; 0 to 5 mg of a colorant, such as ferric oxide; 0 mg to 30 mg, in a present manufacture, 0.1 mg to 30 mg of a hydroxypropylalkylcellulose of 9,000 to 225,000 average-number molecular weight, selected from the group consisting of hydroxypropylethylcellulose, hydroxypropypentylcellulose, hydroxypropylmethylcellulose, and hydropropylbutylcellulose; 0.00 to 1.5 mg of an antioxidant selected from the group consisting of ascorbic acid, butylated hydroxyanisole, butylated hydroxyquinone, butylhydroxyanisole, hydroxycoumarin, butylated hydroxytoluene, cephalm, ethyl gallate, propyl gallate, octyl gallate, lauryl gallate, propyl-hydroxybenzoate, trihydroxybutyrophenone, dimethylphenol, dibutylphenol, vitamin E, lecithin and ethanolamine; and 0.0 mg to 7 mg of a lubricant selected from the group consisting of calcium stearate, magnesium stearate, zinc stearate, magnesium oleate, calcium palmitate, sodium suberate, potassium laurate, salts of fatty acids, salts of alicyclic acids, salts of aromatic acids, stearic acid, oleic acid, palmitic acid, a mixture of a salt of a fatty, alicyclic or aromatic acid and a fatty, alicyclic or aromatic acid.

In the osmotic dosage forms, the semipermeable wall comprises a composition that is permeable to the passage of fluid and impermeable to the passage of compounds disclosed herein. The wall is non-toxic and comprises a polymer selected from the group consisting of a cellulose acylate, cellulose diacylate, cellulose triacylate, cellulose acetate, cellulose diacetate and cellulose triacetate. The wall comprises 75 wt % (weight percent) to 100 wt % of the cellulosic wall-forming polymer; or, the wall can comprise additionally 0.01 wt % to 80 wt % of polyethylene glycol, or 1 wt % to 25 wt % of a cellulose ether selected from the group consisting of hydroxypropylcellulose or a hydroxypropylalkylcellulose such as hydroxypropylmethylcellulose. The total weight percent of all components comprising the wall is equal to 100 wt %. The internal compartment comprises the drug-containing composition alone or in layered position with an expandable hydrogel composition. The expandable hydrogel composition in the compartment increases in dimension by imbibing the fluid through the semipermeable wall, causing the hydrogel to expand and occupy space in the compartment, whereby the drug composition is pushed from the dosage form. The therapeutic layer and the expandable layer act together during the operation of the dosage form for the release of compounds disclosed herein to a subject over time. The dosage form comprises a passageway in the wall that connects the exterior of the dosage form with the internal compartment. The osmotic powered dosage form can be made to deliver drug from the dosage form to the subject at a zero order rate of release over a period of up to about 24 hours.

The expression “passageway” as used herein comprises means and methods suitable for the metered release of the compounds disclosed herein from the compartment of the dosage form. The exit means comprises at least one passageway, including orifice, bore, aperture, pore, porous element, hollow fiber, capillary tube, channel, porous overlay, or porous element that provides for the osmotic controlled release of the compounds disclosed herein. The passageway includes a material that erodes or is leached from the wall in a fluid environment of use to produce at least one controlled-release dimensioned passageway. Representative materials suitable for forming a passageway, or a multiplicity of passageways comprise a leachable poly(glycolic) acid or poly(lactic) acid polymer in the wall, a gelatinous filament, poly(vinyl alcohol), leach-able polysaccharides, salts, and oxides. A pore passageway, or more than one pore passageway, can be formed by leaching a leachable compound, such as sorbitol, from the wall. The passageway possesses controlled-release dimensions, such as round, triangular, square and elliptical, for the metered release of compositions and/or drugs from the dosage form. The dosage form can be constructed with one or more passageways in spaced apart relationship on a single surface or on more than one surface of the wall. The expression “fluid environment” denotes an aqueous or biological fluid as in a human patient, including the gastrointestinal tract. Passageways and equipment for forming passageways are disclosed in Theeuwes et al., U.S. Pat. No. 3,845,770; Theeuwes et al., U.S. Pat. No. 3,916,899; Saunders et al., U.S. Pat. No. 4,063,064; Theeuwes et al., U.S. Pat. No. 4,088,864 and Ayer et al., U.S. Pat. No. 4,816,263. Passageways formed by leaching are disclosed in Ayer et al., U.S. Pat. No. 4,200,098 and Ayer et al., U.S. Pat. No. 4,285,987.

In order to decrease dosing frequency and augment the convenience to the subject and increase subject compliance, the sustained release oral dosage form (regardless of the specific form of the sustained release dosage form) preferably, provides therapeutic concentrations of the compounds disclosed herein in the patient's blood over a period of at least about 6 hours, more preferably, over a period of at least about 8 hours, even preferably, over a period of at least about 12 hours and most preferably, over a period of at least 24 hours.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5 mM to about 50 mM), etc. Additionally, flavoring agents, preservatives, coloring agents, bile salts, acylcarnitines and the like may be added.

Liquid drug formulations suitable for use with nebulizers and liquid spray devices and EHD aerosol devices will typically include compounds disclosed herein with a pharmaceutically acceptable carrier such as, for example, a liquid (e.g., alcohol, water, polyethylene glycol or a perfluorocarbon). Optionally, another material may be added to alter the aerosol properties of the solution or suspension of compositions and/or compounds disclosed herein. In some embodiments, this material is liquid such as an alcohol, glycol, polyglycol or a fatty acid. Other methods of formulating liquid drug solutions or suspension suitable for use in aerosol devices are known to those of skill in the art (Biesalski, U.S. Pat. No. 5,112,598; Biesalski, U.S. Pat. No. 5,556,611).

For topical administration a compound disclosed herein may be formulated as solutions, gels, ointments, creams, suspensions, etc. as are well-known in the art.

For buccal administration, the compounds disclosed herein may take the form of tablets, lozenges, lollipops, etc. formulated in a conventional manner.

Compounds disclosed herein may also be formulated in rectal or vaginal compositions such as suppositories or retention enemas, e.g., containing conventional suppository bases such as cocoa butter or other glycerides.

Systemic formulations include those designed for administration by injection, e.g., subcutaneous, intravenous, intramuscular, intrathecal or intraperitoneal injection, as well as those designed for transdermal, transmucosal, oral or pulmonary administration. Systemic formulations may be made in combination with a further active agent that improves mucociliary clearance of airway mucus or reduces mucous viscosity. These active agents include but are not limited to sodium channel blockers, antibiotics, N-acetyl cysteine, homocysteine and phospholipids.

For injection, compounds disclosed herein may be formulated in aqueous solutions, such as physiologically compatible buffers such as Hanks' solution, Ringer's solution, physiological saline buffer or in association with a surface-active agent (or wetting agent or surfactant) or in the form of an emulsion (as a water-in-oil or oil-in-water emulsion). Suitable surface-active agents include, in particular, non-ionic agents, such as polyoxyethylenesorbitans (e.g., Tween™20, 40, 60, 80 or 85) and other sorbitans (e.g., Span™20, 40, 60, 80 or 85). Compositions with a surface-active agent may comprise between 0.05 and 5% surface-active agent or between 0.1 and 2.5% surface-active agent. The solution may contain formulatory agents such as suspending, stabilizing and/or dispersing agents. Alternatively, compounds disclosed herein may be in powder form for constitution with a suitable vehicle, e.g., sterile pyrogen-free water, before use.

Suitable emulsions may be prepared using commercially available fat emulsions. The combination (or single components) may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. In some embodiments, EDTA is added as a preservative.

In addition to the formulations described previously, compounds disclosed herein may also be formulated as a depot preparation. Such long acting formulations may be administered by implantation (for example, subcutaneously or intramuscularly) or by intramuscular injection. Thus, for example, compounds disclosed herein may be formulated with suitable polymeric or hydrophobic materials (for example, as an emulsion in an acceptable oil) or ion exchange resins, or as sparingly soluble derivatives, for example, as a sparingly soluble salt.

### Therapeutic/Prophylactic Administration And Doses

When used to treat and/or prevent diseases the compounds disclosed herein and/or pharmaceutical compositions thereof may be administered alone or in combination with other pharmaceutical agents including compounds disclosed herein and/or pharmaceutical compositions thereof. The compounds disclosed herein may be administered or applied per se or as pharmaceutical compositions. The specific pharmaceutical composition depends on the desired mode of administration, as is well known to the skilled artisan.

Compounds disclosed herein and/or pharmaceutical compositions thereof may be administered to a subject by intravenous bolus injection, continuous intravenous infusion, oral tablet, oral capsule, oral solution, intramuscular injection, subcutaneous injection, transdermal absorption, buccal absorption, intranasal absorption, inhalation, sublingual, intracerebrally, intravaginally, rectally, topically, particularly to the ears, nose, eyes, or skin or any other convenient method known to those of skill in the art. In some embodiments, compounds disclosed herein and/or pharmaceutical compositions thereof are delivered via sustained release dosage forms, including oral sustained release dosage forms. Administration can be systemic or local. Various delivery systems are known, (e.g., encapsulation in liposomes, microparticles, microcapsules, capsules, “patient controlled analgesia” drug delivery systems, etc.) that can be used to deliver compounds disclosed herein and/or pharmaceutical compositions thereof.

Compounds disclosed herein and/or pharmaceutical compositions thereof may also be administered directly to the lung by inhalation. For administration by inhalation, the compounds disclosed herein and/or pharmaceutical compositions thereof may be conveniently delivered to the lung by a number of different devices. For example, a Metered Dose Inhaler (“MDI”) which utilizes canisters that contain a suitable low boiling propellant, e.g., dichlorodifluoromethane, trichlorofluoromethane, dichlorotetrafluoroethane, carbon dioxide or other suitable gas may be used to deliver the compounds disclosed herein and/or pharmaceutical compositions thereof.

Alternatively, a Dry Powder Inhaler (“DPI”) device may be used to administer compounds disclosed herein and/or pharmaceutical compositions thereof (See, e.g., Raleigh et al., Proc. Amer. Assoc. Cancer Research Annual Meeting, 1999, 40, 397). DPI devices typically use a mechanism such as a burst of gas to create a cloud of dry powder inside a container, which may then be inhaled by the patient. A popular variation is the multiple dose DPI (“MDDPI”) system, which allows for the delivery of more than one therapeutic dose. For example, capsules and cartridges of gelatin for use in an inhaler or insufflator may be formulated containing a powder mix of the compositions and/or compounds disclosed herein and a suitable powder base such as lactose or starch for these systems.

Another type of device that may be used to deliver the compounds disclosed herein and/or pharmaceutical compositions thereof is a liquid spray device supplied, for example, by Aradigm Corporation, Hayward, Calif. Liquid spray systems use extremely small nozzle holes to aerosolize liquid drug formulations that may then be directly inhaled.

In some embodiments, a nebulizer device is used to deliver compounds and/or pharmaceutical compositions thereof disclosed herein. Nebulizers create aerosols from liquid drug formulations by using, for example, ultrasonic energy to form fine particles that may be readily inhaled (e.g., Verschoyle et al., British J. Cancer, 1999, 80, Suppl. 2, 96; Armer et al., U.S. Pat. No. 5,954,047; van der Linden et al., U.S. Pat. No. 5,950,619; van der Linden et al., U.S. Pat. No. 5,970,974).

In still other embodiments, an electrohydrodynamic (“EHD”) aerosol device is used to deliver the compounds disclosed herein and/or pharmaceutical compositions thereof. EHD aerosol devices use electrical energy to aerosolize liquid drug solutions or suspensions (see e.g., Noakes et al., U.S. Pat. No. 4,765,539; Coffee, U.S. Pat. No. 4,962,885; Coffee, International Publication No. WO 94/12285; Coffee, International Publication No. WO 94/14543; Coffee, International Publication No. WO 95/26234; Coffee, International Publication No. WO 95/26235; Coffee, International Publication No. WO 95/32807). Other methods of intra-pulmonary delivery of a compound disclosed herein and/or pharmaceutical composition thereof are known to the skilled artisan and are within the scope of the present disclosure.

Transdermal devices can also be used to deliver the compounds disclosed herein and/or pharmaceutical compositions thereof. In some embodiments, the transdermal device is a matrix type transdermal device (Miller et al., International Publication No. WO 2004/041324). In other embodiments, the transdermal device is a multi-laminate transdermal device (Miller, United States Patent Application Publication No. 2005/0037059).

The amount of compounds disclosed herein and/or pharmaceutical compositions thereof that will be effective in the treatment or prevention of diseases in a patient will depend on the specific nature of the condition and can be determined by standard clinical techniques known in the art. The amount of compounds disclosed herein and/or pharmaceutical compositions thereof administered will, of course, be dependent on, among other factors, the subject being treated, the weight of the subject, the severity of the affliction, the manner of administration and the judgment of the prescribing physician.

### Combination Therapy

In certain embodiments, compounds disclosed herein and/or pharmaceutical compositions thereof can be used in combination therapy with at least one other therapeutic agent. The compounds disclosed herein and/or pharmaceutical compositions thereof and the therapeutic agent can act additively or, more preferably, synergistically. In some embodiments, compounds disclosed herein and/or pharmaceutical compositions thereof are administered concurrently with the administration of another therapeutic agent. For example, compounds disclosed herein and/or pharmaceutical compositions thereof may be administered together with another therapeutic agent (e.g. including, but not limited to, peripheral opioid antagonists, laxatives, non-opioid analgesics and the like). In other embodiments, compounds disclosed herein and/or pharmaceutical compositions thereof are administered prior or subsequent to administration of other therapeutic agents.

It will be apparent to those skilled in the art that many modifications, both to materials and methods, may be practiced without departing from the scope of this disclosure.

Accordingly, the present embodiments are to be considered as illustrative and not restrictive, and the invention is not to be limited to the details given herein, but may be modified within the scope and equivalents of the allowed claims.

All publications and patents cited herein are incorporated by reference in their entirety.

The following examples illustrate the invention.

In the examples, the following abbreviations are used: —

HOBt: 1-Hydroxybenzotriazole; PyBOP: Benzotriazole-1-yl-oxy-tris-pyrrolidino-phosphonium hexafluorophosphate; DIEA: diisopropylethylamine; and BocGlyOSu: N—(N-alpha-glycinyloxy)succinimide.

Amino acids in structures depicted in the examples are intended to be natural L amino acids. Each structure can be any possible stereoisomer or any mixture thereof.

### Preparation 1

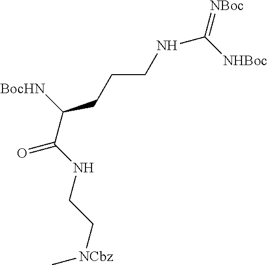

BocArg(diBoc)OH (Bachem, 0.47 g, 1.0 mmol) was dissolved in dimethylformamide (5 ml) and mixed with HOBt (0.15 g, 1.15 mmol) and PyBOP (0.6 g, 1.15 mmol). Diisopropylethylamine (0.4 ml, 2.3 mmol) was added to the mixture, then the resulting solution was stirred for 10 minutes and added to a solution of H₂NCH₂CH₂N(CH₃)CBz (0.28 g, 1.15 mmol) in dimethylformamide (3 ml). The basicity was adjusted by addition of DIEA (0.4 ml, 2.3 mmol). The mixture was stirred for 2 hours and then poured into 40 ml of 5% aqueous citric acid. The product was extracted with a 20 ml of ethyl ether and ethyl acetate (5:1). The organic layer was washed with water, two times with 10 ml of 1M aqueous sodium carbonate, water and brine, and then dried over magnesium sulfate. The solvents were removed by evaporation to afford the depicted product (0.65 g, 98%).

### Preparation 2

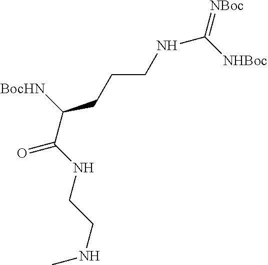

The product of Preparation 1(0.65 g, 0.98 mmol) was dissolved in ethanol (10 ml). Pearlman's catalyst (0.32 g) was then added and the mixture was subjected to hydrogenation (1 atm, about 101 kPa, 24 h). The resultant mixture was then filtered from the catalyst and the solvent was removed by evaporation. The residue was further dried under high vacuum for 2 hours to afford the depicted product (0.525 g, 99%).

### Preparation 3

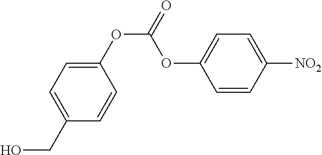

A solution of p-nitrophenylchlorocarbonate (2.0 g, 10 mmol) in tetrahydrofuran (10 ml) was added to a solution of 4-hydroxymethylphenol (1.24 g, 10 mmol) and triethylamine (1.4 ml, mmol) in tetrahydrofuran (10 ml). The mixture was stirred for 2 hours. A precipitate formed and was filtered out. The filtrate was then evaporated to dryness. The residue was partitioned between ethyl acetate and water. The organic layer was washed with water and brine, then dried over magnesium sulfate. The solvent was then removed by evaporation and the residue was triturated with dichloromethane (10 ml). A white precipitate formed, and this was filtered off, washed with dichloromethane (5 ml) and dried in air to afford the depicted product (2.1 g, 75%).

### Preparation 4

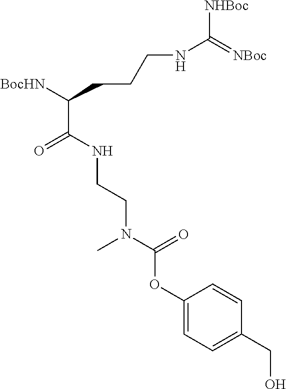

The product of Preparation 3 (0.15 g, 0.5 mmol) and the product of Preparation 2 (0.34 g, 0.5 mmol) were dissolved in dichloromethane (10 ml). Triethylamine (0.077 ml, 0.55 mmol) was then added and the reaction mixture was stirred for 24 hours. The solvent was then removed by evaporation and the residue was partitioned between ethyl acetate (15 ml) and 5% aqueous citric acid (20 ml). The organic layer was washed with water, four times with 1M aqueous sodium carbonate (10 ml), water and brine, and then dried over magnesium sulfate. The solvent was removed by evaporation to afford the depicted product (0.28 g, 82%).

### Preparation 5

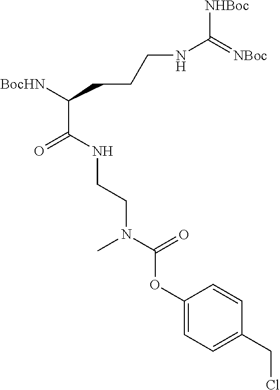

The product of Preparation 4 (0.28 g, 0.41 mmol) was dissolved in dichloromethane (10 ml), containing 2,6-lutidine (1 ml). Thionyl chloride (0.085 g, 0.56 mmol) was then added and the resultant mixture was stirred for 2 hours. The solvents were then removed by evaporation and the residue was partitioned between 5% aqueous citric acid (10 ml) and ethyl acetate (10 ml). The organic layer was then washed with water and brine, then dried over magnesium sulfate. The solvent was then removed by evaporation and the residue (the depicted product, 0.24 g, 84%) was used in the next step without further purification.

### Preparation 6

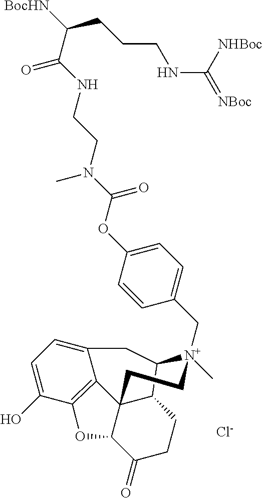

The product of Preparation 5 (0.24 g, 0.34 mmol), hydromorphone (0.12 g, 0.43 mmol) and lithium bromide (4.3 mg, 0.05 mmol) were dissolved in dimethylformamide (3 ml) and stirred for 24 hours. The solvent was then removed by evaporation under a high vacuum and the residue was triturated with diethyl ether (5 ml) to afford the depicted product (0.31 g, 100%).

### Example 1

### Hydromorphone N-(4-(N′-methyl-N′-(2-arginylaminoethyl)aminocarbonyloxy)-phenylmethyl Chloride

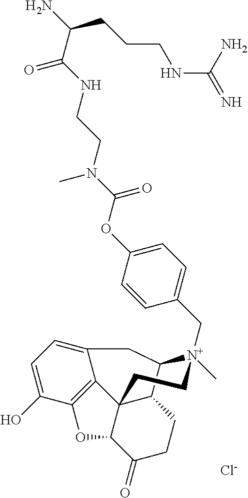

The product of Preparation 6 (0.31 g, 0.34 mmol) was dissolved in a 1:1 mixture of trifluoroacetic acid and dichloromethane (6 ml). The reaction mixture was then stirred for 6 hours. The solvents were then removed by evaporation under a vacuum and the residue was triturated three times with anhydrous diethyl ether. The residue was dissolved in 0.1N hydrochloric acid (5 ml) and purified by reverse phase preparative HPLC (acetonitrile gradient) to afford the depicted compound (0.058 g, 22.5%). Mass spec: Calculated 683.3. Observed 684.5.

### Example 2

### Hydromorphone N-(4-(N′-methyl-N′-(2-N″-acetylarginylaminoethyl)-aminocarbonyloxy)phenylmethyl chloride

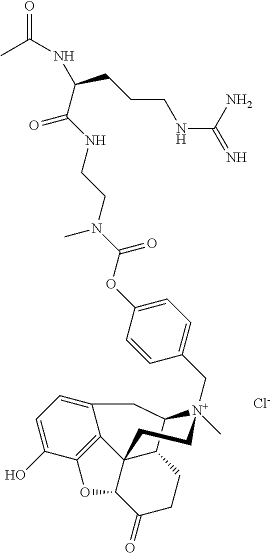

The product of Example 1 (0.034 g, 0.044 mmol) was dissolved in a 1M aqueous sodium carbonate solution (3 ml) and acetic anhydride (0.45 g, 0.44 mmol) was added portionwise over a period of 2 h. The reaction mixture was stirred for 4 h and then acidified by careful addition of 1N aqueous hydrochloric acid. The resultant solution was then purified by reverse phase preparative HPLC (acetonitrile gradient) to afford the depicted product (0.013 g, 50%). Mass spec: Calculated 725.3. Observed 726.3.

### Example 3

### Hydromorphone N-(4-(N′-methyl-N′-(2-arginyl(methyl)aminoethyl)aminocarbonyloxy)-phenylmethyl Chloride

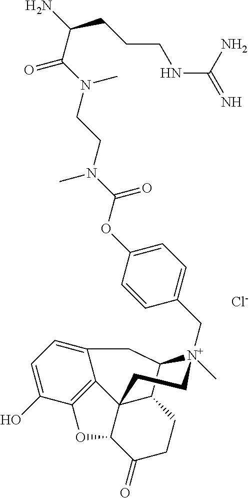

Prepared following the method of Preparations 1 to 6 and Example 1, but using CH₃NHCH₂CH₂N(CH₃)CBz instead of H₂NCH₂CH₂N(CH₃)CBz. Mass spec: Calculated 697.4. Observed 698.3.

### Example 4

### Hydromorphone N-(4-(N′-methyl-N′-(2-prolinylaminoethyl)aminocarbonyloxy)-phenylmethyl Chloride

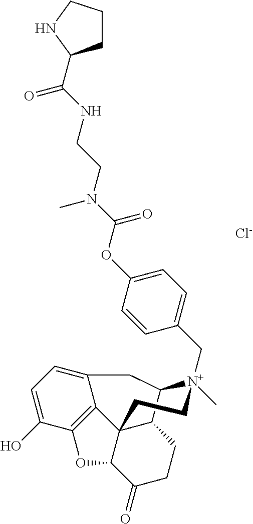

Prepared following the method of Preparations 1 to 6 and Example 1, but using N-Boc L-proline instead of BocArg(diBoc)OH. Mass spec: Calculated 488.3. Observed 487.9.

### Example 5

### Hydromorphone N-(4-(N′-methyl-N′-(2-(N″-glycinyl)prolinylaminoethyl)-aminocarbonyloxy)phenylmethyl Chloride

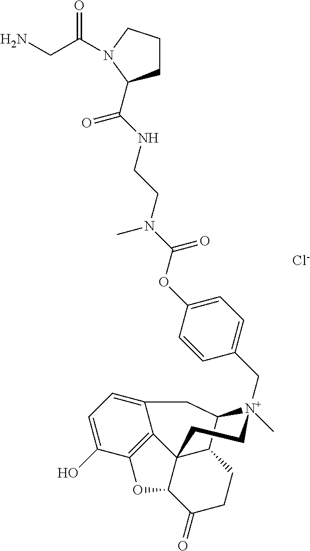

Prepared by the method of Example 2 starting from the compound of Example 4, but using BocGlyOSu instead of acetic anhydride. Mass spec: Calculated 545.3. Observed 545.3.

### Preparation 7

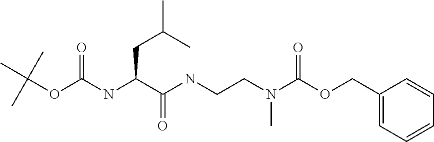

Boc-L-Leu-OH hydrate (3 mmol, 0.75 g), H₂NCH₂CH₂N(CH₃)CBz hydrochloride (3.1 mmol 0.75 g) and PyBOP (3.1 mmol, 1.33 g) were dissolved in 25 ml DMF, then DIEA (1.2 ml, ˜7 mmol) was added. The reaction mixture was stirred 2 h at ambient temperature, then diluted with 100 ml ethyl acetate and transferred to a separatory funnel. The ethyl acetate layer was washed twice with water (2×150 ml) and brine (100 ml), then dried over MgSO₄. The drying agent was filtered off and the solvent was removed under reduced pressure to afford the depicted product (˜1.2 g, 95%).

### Preparation 8

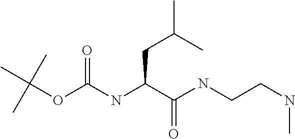

The product of Preparation 7 (1.2 g, 2.9 mmol) was dissolved in ˜30 ml MeOH. The reaction vessel was purged with nitrogen, then 5% Pd/C (˜150 mg) was added. Hydrogenolysis was performed at 60 psi (about 414 kPa) during 2 h on a Parr's apparatus. The catalyst was then filtered off and the solvent was removed under reduced pressure to afford the depicted product as a clear oil (0.84 g, ˜100%).

### Preparation 9

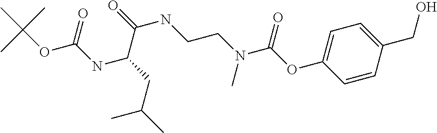

The product of Preparation 8 (0.84 g, 2.9 mmol) and the product of Preparation 3 were dissolved in DMF (15 ml). The reaction mixture was kept at ambient temperature for 3 h, then diluted with ethyl acetate (100 ml) and transferred to separatory funnel. The organic layer was washed twice with water (2×150 ml) and brine (100 ml), then dried over MgSO₄ and evaporated. The crude yield of the depicted product was ˜1 g (˜80%).

### Preparation 10

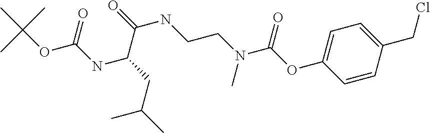

The product of Preparation 5 (1 g, 2.3 mmol) and DIEA (0.7 ml, ˜4 mmol) were dissolved in chloroform (20 ml). The reaction mixture was cooled to around −30 to −40° C., then SOCl₂ (0.3 ml, ˜4 mmol) was added dropwise. The reaction mixture was warmed up to room temperature (1 h) and directly loaded on a silica gel column (combiflash, 40 g column). The fractions containing the depicted product were pooled out, evaporated and re-purified by RP HPLC on C18 (1.5 in.×300), yielding 0.43 g (41%) of the depicted product with a purity of more than 95% (LC-MS).

### Preparation 11

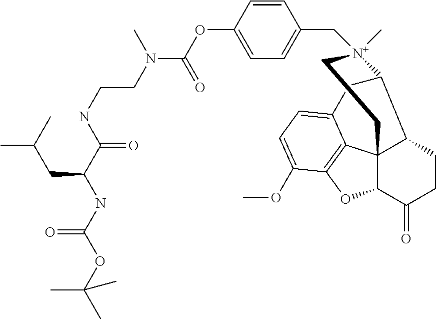

Hydroxycodone (free base) (0.3 g, 1 mmol) and the product of Preparation 10 (0.43 g, 0.95 mmol) were dissolved in DMF (7 ml) followed by addition of LiBr (0.1 g, 1.1 mmol). The reaction solution turned yellow. The reaction mixture was kept at ambient temperature for 40 h. Conversion by LC MS was ˜60-70%. DMF solution was loaded onto a RP HPLC column (1.5 in ×300). RP chromatography was performed in an acetonitrile gradient (10-90%) 90 min, 50 ml/min. The fractions containing the depicted product were collected and evaporated.

### Example 6

### Hydrocodone N-(4-(N′-methyl-N′-(2-L-leucinylaminoethyl)aminocarbonyloxy)-phenylmethyl Chloride

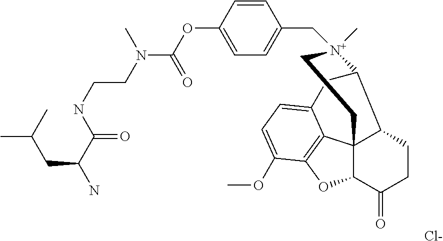

The product of Preparation 11 was treated for 5 min with 15 ml TFA at ambient temperature. The acid was then evaporated. The residual oil was dissolved in ˜4 ml AcOH, diluted with ml 2M HCl in ether and 30 ml ether. Solid material was precipitated by centrifugation, treated with ether again and dried under high vacuum overnight. Yield: 0.09 g, (15%). Purity: 97%. Mass spec: Calculated 619.79. Observed 619.6.

### Preparation 12

### Synthesis of (2-Benzyloxycarbonylamino-ethyl)methylcarbamic acid 9H-fluoren-9-ylmethyl Ester

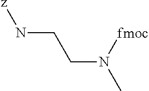

A mixture of sodium bicarbonate (NaHCO₃) (0.84 g, 10 mmol), Fmoc-OSu (2.44 g, 10 mmol) and the hydrochloric acid salt of (2-methylamino-ethyl)-carbamic acid 9H-fluoren-9-ylmethyl ester (2.44 g, 10 mmol) in acetonitrile/water (3:1, 250 ml) was stirred at ambient temperature for 2 h. The solvents were then evaporated under a vacuum. The residue was dissolved in ethyl acetate (EtOAc) (150 ml) and washed with water 3 times (50 ml each) and brine (50 ml). The organic layer was dried over magnesium sulfate (MgSO₄) and evaporated under a vacuum. The residue was dried under a vacuum overnight at ambient temperature to provide the title compound (3.91 g, 91%) as off-white solid. Mass spec: Calculated 431.5. Observed 431.5.

### Preparation 13

### Synthesis of (2-Amino-ethyl)-methyl-carbamic acid 9H-fluoren-9-ylmethyl Ester

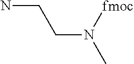

The product of Preparation 12 (3.91 mg, 9.1 mmol) was dissolved in methanol (50 ml) followed by the addition of hydrochloric acid (HCl) (conc., 1.5 ml, 18 mmol) and palladium on carbon (Pd/C) (5% wt, 1 g) suspension in water (2 ml). The reaction mixture was subjected to hydrogenation (Parr apparatus, 60 psi) at ambient temperature for 30 min. The catalyst was filtered off and washed with methanol. The filtrate was evaporated with isopropanol (i-PrOH) (2×20 ml) under a vacuum. The residue was dried under a vacuum overnight to provide the hydrochloric salt of the title compound (3.01 g, 99%) as white solid. Mass spec: Calculated 297.4. Observed 297.5.

### Preparation 14

### Synthesis of Carbonic Acid 4-hydroxymethylphenyl Ester 4-nitro-phenyl Ester

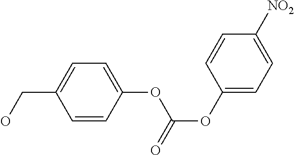

A mixture of 4-hydroxybenzyl alcohol (7.43 g, 60 mmol), 4-nitrophenyl chloroformate (12.1 g, 60 mmol) and DIEA (10.5 ml, 60 mmol) in chloroform (300 ml) was stirred at ambient temperature for 1 h. The reaction mixture was evaporated. The residue was dissolved in ethyl acetate (200 ml) and extracted with water 3 times (70 ml each) and brine (70 ml), and dried over MgSO₄. The solvent was evaporated in vacuum. The residue was dissolved in chloroform (100 ml) and subjected to flash chromatography (glass column 80×200 mm with glass frit) on Silica gel (Merck, grade 60, 70-230 mesh, 60A) in step gradient mode (washing 100% chloroform following 1% methanol (MeOH)/chloroform; elution at 2% MeOH/chloroform). Fractions containing the desired product were collected and evaporated. The residue was dried in vacuum overnight to produce the title compound (7.6 g, 44%) as white solid. Mass spec: Calculated 368.3. Observed 368.1.

### Example 7

### Hydrocodone N-(4-(N′-methyl-N′-(2-arginylaminoethyl)aminocarbonyloxy)-phenylmethyl Chloride

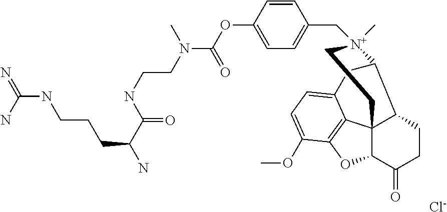

Step 1. A mixture of H-Arg(Pbf)-OH (854 mg, 2.0 mmol) and BSA (bis(trimethysilyl)acetamide—1.08 ml, 4.4 mmol) in dimethylformamide (DMF) (6 ml) was stirred at ambient temperature overnight.

Step 2. p-Nitrophenylcarbonate Wang resin (1.15 g, 1.0 mmol; Novabiochem®, 0.87 mmol/g, 100-200 mesh) was placed in a 20 cc plastic syringe equipped with plunger and plastic frit. The resin was swelled in dichloromethane (DCM) (10 ml) at ambient temperature for 10 min. DCM was discharged followed by resin swelling twice in DMF (10 ml for 10 min each). The solvent was discharged and the resin was treated with the combined solution from step 1 and dimethylaminopyridine (DMAP) (244 mg, 2.0 mmol). The syringe was agitated at 160 rpm on an orbital shaker at ambient temperature overnight. The solution was discharged and the resin was washed with DMF (four times, each with 10 ml for 5 min), methanol (twice, each with 10 ml for 5 min) and DMF (twice, each with 10 ml for 5 min) followed by the addition of the solution of the product of Preparation 13 (444 mg, 1.5 mmol), BOP (665 mg, 1.5 mmol), 1-hydroxybenzotriazole (HOBt) (203 mg, 1.5 mmol) and DIEA (522 μl, 3.0 mmol) in DMF (6 ml). The syringe was agitated at 160 rpm on an orbital shaker at ambient temperature overnight. The solution was discharged and the resin was washed with DMF (four times, each with 10 ml for 5 min) followed by the treatment with 20% piperidine/DMF (twice, each with 10 ml for 10 min). The solvents were discharged and the resin was washed with DMF (six times, each with 10 ml for 5 min) followed by the addition of the solution of the product of Preparation 14 (434 mg, 1.5 mmol) and HOBt (270 mg, 2.0 mmol) in DMF (6 ml). The syringe was agitated at 160 rpm on an orbital shaker at ambient temperature overnight. The solution was discharged and the resin was washed with DMF (six times, each with 10 ml for 5 min) followed by the addition of a solution of methanesulfonyl chloride (MsCl) (390 μl, 5.0 mmol) and DIEA (957 μl, 5.5 mmol) in DMF (6 ml). The syringe was agitated at 160 rpm on an orbital shaker at ambient temperature overnight. The solution was discharged and the resin was washed with DMF (six times, each with 10 ml for 5 min) followed by the addition of a solution of hydrocodone (free base, 449 mg, 1.5 mmol) and lithium bromide (LiBr) (261 mg, 3.0 mmol) in DMF (6 ml). The syringe was agitated at 160 rpm on an orbital shaker at ambient temperature overnight. The solution was discharged and the resin was washed with DMF (six times, each with 10 ml for 5 min), methanol (twice, each with 10 ml for 10 min) and ether (twice, each with 10 ml for 10 min). The resin was dried in vacuum at ambient temperature for 4 h followed by addition of 5% m-cresol/trifluoroacetic acid (TFA) (10 ml). The syringe was agitated at 160 rpm on an orbital shaker at ambient temperature for 1 h. The supernatant was distributed in 4 plastic centrifuge tubes (50 cc) equipped with plastic stoppers. Ether (45 ml in each tube) was added. The formed precipitate was centrifuged. The supernatant was discharged. The aforementioned procedure was repeated one more time. The precipitate was air dried, dissolved in water (10 ml) and subjected to high performance liquid chromatography (HPLC) purification. [Nanosyn-Pack Microsorb (100-10) C-18 column (50×300 mm); flow rate=100 ml/min; injection volume 10 ml; mobile phase A: 100% water, 0.1% TFA; mobile phase B: 100% acetonitrile (ACN), 0.1% TFA; gradient elution from 0% B to 70% B in 70 min., detection 254 nm]. Fractions containing the desired compound were combined and concentrated in vacuum. The residue was dissolved in isopropanol (15 ml) and evaporated in vacuum to provide a TFA salt of the title compound as colorless glass-like solid. The TFA salt of the title compound was dissolved in dioxane (2 ml) and 4 M HCl/dioxane (10 ml) was added. The reaction mixture was maintained at ambient temperature for 30 min. Solvents were evaporated. The residue was dried in vacuum to provide hydrochloric acid salt of the title compound as white solid. Yield: 67 mg (10%). Purity: 98%. Mass spec: Calculated 662.8. Observed 662.3.

### Example 8

### Hydrocodone N-(4-(N′-methyl-N′-(2-alaninylaminoethyl)aminocarbonyloxy)-phenylmethyl Chloride

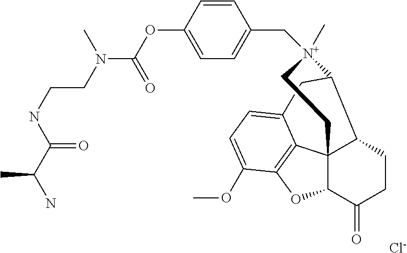

Prepared following the method described in Example 7, but starting from the appropriate amino acid-containing starting material. The method provided a hydrochloric salt of the title compound as white solid. Yield: 29 mg. Purity: 99.2%. Mass spec. Calculated: 577.7. Observed 577.2.

### Example 9

### Hydrocodone N-(4-(N′-methyl-N′-(2-aspartylaminoethyl)aminocarbonyloxy)-phenylmethyl Chloride

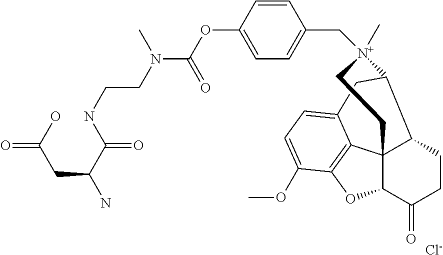

Prepared following the method described in Example 7, but starting from the appropriate amino acid-containing starting material. The method provided a hydrochloric salt of the title compound as white solid. Yield: 57 mg. Purity: 99.9%. Mass spec. Calculated: 621.7. Observed 621.2.

### Example 10

### Hydrocodone N-(4-(N′-methyl-N′-(2-tyrosinylaminoethyl)aminocarbonyloxy)-phenylmethyl Chloride

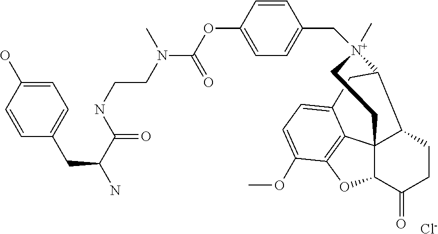

Prepared following the method described in Example 7, but starting from the appropriate amino acid-containing starting material. The method provided a hydrochloric salt of the title compound as white solid. Yield: 57 mg. Purity: 99.9%. Mass spec. Calculated: 670.0. Observed 669.2.

### Example 11

### Hydrocodone N-(4-(N′-methyl-N′-(2-lysinylaminoethyl)aminocarbonyloxy)-phenylmethyl Chloride

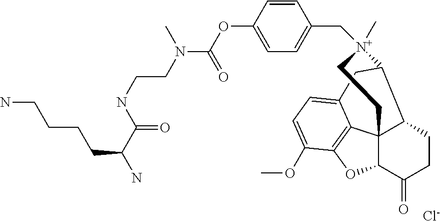

Prepared following the method described in Example 7, but starting from the appropriate amino acid-containing starting material. The method provided a hydrochloric salt of the title compound as white solid. Yield: 52 mg. Purity: 97.3%. Mass spec. Calculated: 634.8. Observed 634.4.

### Preparation 15

### Fmoc-Gly-Arg(Pbf)-OH

Step 1. A solution of Fmoc-Gly-OH (1.14.84 g, 3.85 mmol), BOP (2.05 g, 4.62 mmol) and DIEA (1.47 ml, 8.47 mmol) was stirred at ambient temperature for 20 min.

Step 2. A mixture of H-Arg(Pbf)-OH (1.65 g, 3.85 mmol) and BSA (2.08 ml, 8.47 mmol) in DMF (3 ml) was stirred at ambient temperature for 20 min followed by the addition of solution obtained in step 1. The reaction mixture was maintained at ambient temperature for min followed by dilution with ethyl acetate (150 ml) and washing with water (three times, each with 50 ml) and brine (50 ml). The organic layer was dried over MgSO₄ and evaporated in vacuum. The residue was dried in vacuum overnight at ambient temperature to provide the title compound (2.50 g, 92%) as yellowish solid. Mass spec: Calculated 706.8. Observed 706.5.

### Preparation 16

### Synthesis of H-Gly-Arg(Pbf)-OH

A solution of the product of Preparation 15 (2.5 g, 3.54 mmol) and piperidine (1.75 ml, 17.7 mmol) in ethyl acetate (10 ml) was stirred at ambient temperature for 40 min. Solvents were evaporated. The residue was triturated with hexane. The formed precipitate was filtrated and washed with hexane. The precipitate was dissolved in ethyl acetate (10 ml) and hexane was added (300 ml). The formed precipitate was filtered and washed with hexane. The aforementioned procedure was repeated one more time. The residue was dried under a vacuum overnight at ambient temperature to provide the title compound (0.98 g, 57%) as yellowish solid. Mass spec: Calculated 484.6. Observed: 484.5.

### Example 12

### Hydrocodone N-(4-(N′-methyl-N′-(2-glycinylarginylaminoethyl)aminocarbonyloxy)-phenylmethyl Chloride

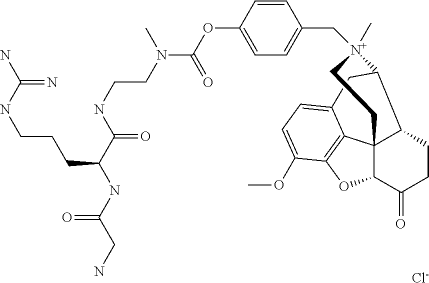

Step 1. A mixture of the product of Preparation 16 (966 mg, 2.0 mmol) and BSA (1.08 ml, 4.4 mmol) in DMF (6 ml) was stirred at ambient temperature overnight.

Step 2. p-Nitrophenylcarbonate Wang resin (1.15 g, 1.0 mmol; Novabiochem®, 0.87 mmol/g, 100-200 mesh) was placed in a 20 cc plastic syringe equipped with plunger and plastic frit. The resin was swelled in DCM (10 ml) at ambient temperature for 10 min. DCM was discharged followed by resin swelling twice in DMF (each with 10 ml for 10 min). The solvent was discharged and the resin was treated with the combined solution from step 1 and DMAP (244 mg, 2.0 mmol). The syringe was agitated at 160 rpm on an orbital shaker at ambient temperature overnight. The solution was discharged and the resin was washed with DMF (four times, each with 10 ml for 5 min), methanol (twice, each with 10 ml for 5 min) and DMF (twice, each with 10 ml for 5 min) followed by the addition of the solution of the product of Preparation 13 (444 mg, 1.5 mmol), BOP (665 mg, 1.5 mmol), HOBt (203 mg, 1.5 mmol) and DIEA (522 μl, 3.0 mmol) in DMF (6 ml). The syringe was agitated at 160 rpm on an orbital shaker at ambient temperature overnight. The solution was discharged and the resin was washed with DMF (four times, each with 10 ml for 5 min) followed by treatment with 20% piperidine/DMF (twice, each with 10 ml for 10 min). The solvents were discharged and the resin was washed with DMF (six times, each with 10 ml for 5 min) followed by the addition of the solution of the product of Preparation 14 (434 mg, 1.5 mmol) and HOBt (270 mg, 2.0 mmol) in DMF (6 ml). The syringe was agitated at 160 rpm on an orbital shaker at ambient temperature overnight. The solution was discharged and the resin was washed with DMF (six times, each with 10 ml for 5 min) followed by the addition of a solution of MsCl (390 μl, 5.0 mmol) and DIEA (957 μl, 5.5 mmol) in DMF (6 ml). The syringe was agitated at 160 rpm on an orbital shaker at ambient temperature overnight. The solution was discharged and the resin was washed with DMF (six times, each with 10 ml for 5 min) followed by addition of a solution of hydrocodone (free base, 449 mg, 1.5 mmol) and LiBr (261 mg, 3.0 mmol) in DMF (6 ml). The syringe was agitated at 160 rpm on an orbital shaker at ambient temperature overnight. The solution was discharged and the resin was washed with DMF (six times, each with 10 ml for 5 min), methanol (twice, each with 10 ml for 10 min) and ether (twice, each with 10 ml for 10 min). The resin was dried in vacuum at ambient temperature for 4 h followed by addition of 5% m-cresol/TFA (10 ml). The syringe was agitated at 160 rpm on an orbital shaker at ambient temperature for 1 h. The supernatant was distributed in 4 plastic centrifuge tubes (50 cc) equipped with plastic stoppers. Ether (45 ml in each tube) was added. The formed precipitate was centrifuged. The supernatant was discharged. The aforementioned procedure was repeated one more time. The precipitate was air dried, dissolved in water (10 ml) and subjected to HPLC purification. [Nanosyn-Pack Microsorb (100-10) C-18 column (50×300 mm); flow rate=100 ml/min; injection volume 10 ml; mobile phase A: 100% water, 0.1% TFA; mobile phase B: 100% ACN, 0.1% TFA; gradient elution from 0% B to 70% B in 70 min., detection 254 nm]. Fractions containing the desired compound were combined and concentrated in vacuum. The residue was dissolved in isopropanol (15 ml) and evaporated in vacuum to provide a TFA salt of the title compound as colorless glass-like solid. The TFA salt of the title compound was dissolved in dioxane (2 ml) and 4 M HCl/dioxane (10 ml) was added. The reaction mixture was maintained at ambient temperature for 30 min. Solvents were evaporated. The residue was dried in vacuum to provide hydrochloric salt of the title compound as white solid. Yield: 42 mg (6%). Purity: 97%. Mass spec: Calculated 719.9. Observed 719.5.

### Example 13

### Hydrocodone N-(4-(N′-methyl-N′-(2-L-asparaginylaminoethyl)aminocarbonyloxy)-phenylmethyl Chloride

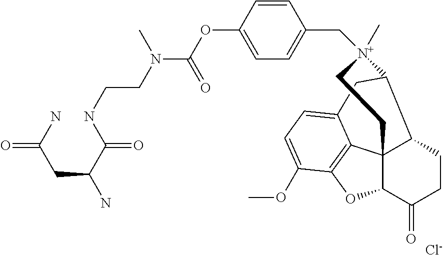

Prepared following the method described in Example 7, but starting from the appropriate amino acid-containing starting material. The method provided a hydrochloric salt of the title compound as white solid. Yield: 50 mg (8%). Purity: 98.4%. Mass spec. Calculated: 620.7. Observed 620.4.

Following the methods of the Preparations and Examples described hereinabove, the following compounds may also be prepared, and are provided as further embodiments of the invention:—

### 1. Hydrocodone N-(4-(N′-methyl-N′-(2-N″-acetyl-L-arginylaminoethyl)-aminocarbonyloxy)phenylmethyl Chloride

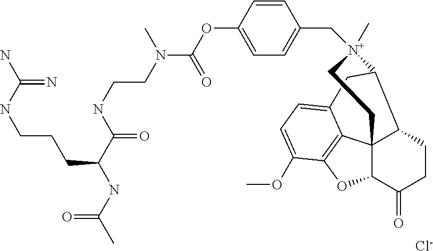

### 2. Hydrocodone N-(4-(N′-methyl-N′-(2-N″-acetylglycinyl-L-arginylaminoethyl)-aminocarbonyloxy)phenylmethyl Chloride

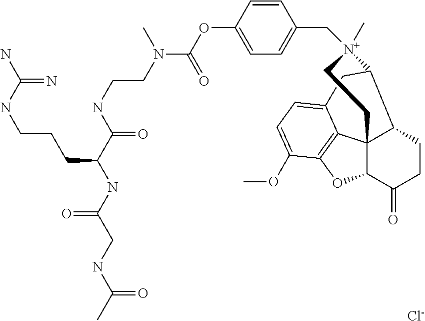

### 3. Hydrocodone N-(4-(N′-methyl-N′-(2-N″-acetyl-L-alaninylaminoethyl)-aminocarbonyloxy)phenylmethyl Chloride

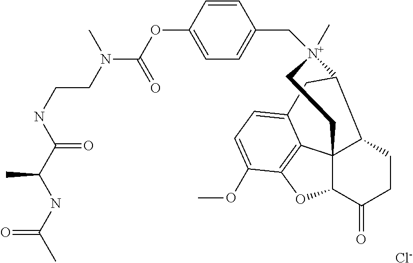

### 4. Hydrocodone N-(4-(N′-methyl-N′-(2-N″-acetyl-L-asparaginylaminoethyl)-aminocarbonyloxy)phenylmethyl Chloride

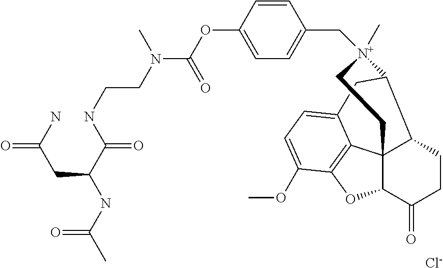

### 5. Hydrocodone N-(4-(N′-methyl-N′-(2-N″-acetyl-L-aspartylaminoethyl)-aminocarbonyloxy)phenylmethyl Chloride

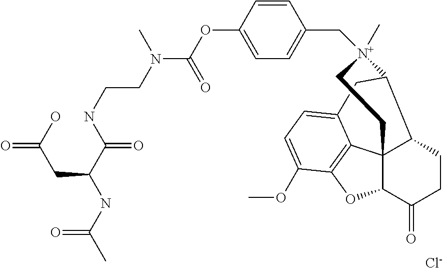

### 6. Hydrocodone N-(4-(N′-methyl-N′-(2-N″-acetyl-L-tyrosinylaminoethyl)-aminocarbonyloxy)phenylmethyl Chloride

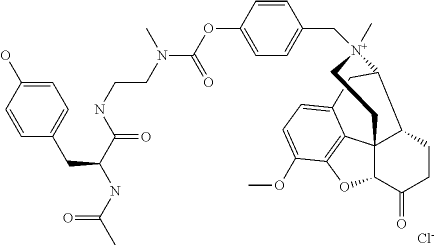

### 7. Hydrocodone N-(4-(N′-methyl-N′-(2-N″-acetyl-L-lysinylaminoethyl)-aminocarbonyloxy)phenylmethyl Chloride

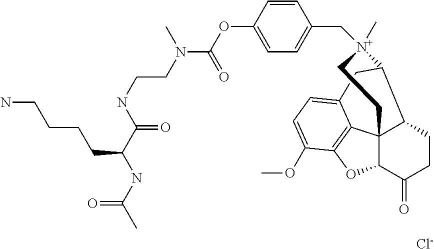

### 8. Hydrocodone N-(4-(N′-methyl-N′-(2-N″-acetyl-L-leucinylaminoethyl)-aminocarbonyloxy)phenylmethyl Chloride

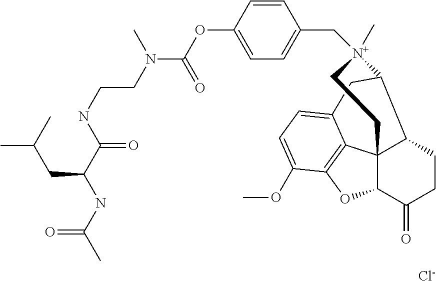

### Protocols for Evaluating Test Compounds

1a. “Kitchen” Test

The stability of a test compound in the presence of the readily available household chemicals, acetic acid (vinegar) and sodium bicarbonate (baking soda) may be demonstrated in the following “Kitchen” Test.

0.5 mg of a test compound is dissolved in 1 ml of each of the following solutions corresponding with possible household chemicals: 30% aqueous acetic acid; 50% aqueous ethanol and saturated aqueous solution of sodium bicarbonate (baking soda). Each solution is kept at room temperature for 20-24 hours and then heated at 85° C. for 20-24 hours. Hydromorphone release and general stability are monitored by analytical HPLC. A compound is considered as having passed this test if after 20 hours the hydromorphone concentration does not exceed 10% of the starting material or other product of degradation.

All compounds exemplified herein that have been subjected to this test have passed.

1b. Demonstration of the controlled release of parent drug from “activated” prodrugs.

### In Vitro Demonstration

The controlled release of parent drug (e.g. hydromorphone) from the prodrug was demonstrated by the synthesis, and in vitro testing of Compound A depicted in Table 1. By design, the intramolecular cyclization-release sequence results in the concomitant formation of a cyclic urea with the release of the parent drug.

These release kinetics of this compound were evaluated at increasing pH. The liberation of hydromorphone during the course of these reactions was confirmed by LC-MS analysis. It is interesting to note that the intramolecular cyclization-release reactions can be suppressed at low pH by deactivation of the nucleophilic nitrogen atom via protonation. Compounds of the invention in which R⁴ is an amino acid residue are stable under these conditions since the nucleophilic nitrogen has been acylated by the amino acid residue. These data strongly support the functional roles of the spacer and the “activated” nucleophilic nitrogen in the intramolecular cyclization-release of the parent drug molecule.

### 2. In Vitro Human μ-Opioid Receptor Binding Assay.

TABLE 1
The liberation of hydromorphone from prodrug in base.

| | % | | |
|---|---|---|---|
| | production | | |
| | of hydro- | | |
| | morphone | | |
| | 20 hrs | | |
| | pH | | |
| Structure | 7.5 | 10 | cpd. |
| 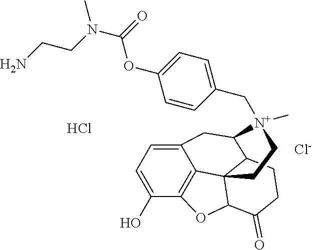 | 70 | 100 | A |

This test measures the affinity of test compounds for the t-opioid receptor relative to hydromorphone.

### General Procedure:

The general procedure follows the protocol described by Wang, J.-B., Johnson, P. S., Perscio, A. M., Hawkins, A. L., Griffin, C. A. and Uhl, G. R. (1994). FEBS Lett., 338: 217-222.
Assay: t-opioid receptor
Origin: human recombinant (HEK-293 cells)
Reference compound: [d-Ala2,N-Me-Phe⁴,Gly⁵⁻ol]-enkephalin (DAMGO)
Radioligand: [³H]DAMGO (0.5 nM)
Non-specific ligand: naloxone (10 uM)
Incubation: 120 min@22° C.
Method of detection: scintillation counting

Analysis and expression of results: The specific binding to the receptors is defined as the difference between the total binding and the non-specific binding determined in the presence of an excess of unlabelled ligand. The results depicted in Table 2 are expressed as a percent of control of specific binding and as a present inhibition of control specific binding obtained in the presence of test compounds. The IC₅₀ values (molar concentration causing a half-maximal inhibition of control specific binding), and Hill coefficients (nH) were determined by non-linear regression analysis of competition curves using Hill equation curve fitting.

Results

TABLE 2

| Example | IC₅₀ HU μ-opioid receptor |
|---|---|
| Hydromorphone HCl (HM) | 1E−09 |
| 1 | 2.5E−07 |

The above results are consistent with the structure activity relationships for opioids obtained in the literature, obtained from screening of these representative molecules, demonstrate the deactivation of opioid potency when the promoiety is appended to either the phenol or tertiary nitrogen residues of hydromorphone.

### 3. Pharmacokinetic Data

### Plasma Timecourse of Hydromorphone or Hydrocodone Following IV or Oral Administration in Rat

IV dosing: Test compound is dissolved in saline (2 mg/ml) and injected into the tail vein of jugular vein cannulated male Sprague-Dawley rats. Hydromorphone (HM) or Hydrocodone (HC), respectively, at 1 mg/kg is used as a positive control, and test compound is dosed, for HM-containing compounds, at a parent opioid equivalent dose equal to 1 mg/kg and for HC-containing compounds, at 2 mg/ml. At specified time points, blood is withdrawn, quenched into methanol, centrifuged at 14000 rpm@4° C., and stored at −80° C. until analysis. Samples are quantified via LC/MS/MS using an ABI 3000 triple-quad mass spectrometer.

Oral dosing: The test compound is dissolved in saline (20 mg/ml) and dosed via oral gavage into jugular vein cannulated male Sprague-Dawley rats. HM or HC, respectively, at 10 mg/kg is used as a positive control and the test compound is dosed, for HM-containing compounds, at an approximate parent opioid equivalent (equimolar) dose equal to 10 mg/kg, and for HC-containing compounds, at the doses indicated in Table 6. At specified time points, blood is withdrawn, quenched into methanol, centrifuged at 14000 rpm@4° C., and stored at −80° C. until analysis. Samples are quantified via LC/MS/MS using an ABI 3000 triple-quad mass spectrometer.

Results:

TABLE 3
Maximum concentration (Cmax) of hydromorphone
(HM) found in blood after IV dosing in rats.

| Example | Cmax HM (ng/ml) following IV dosing |
|---|---|
| Hydromorphone | 352 |
| 1 | 38 |
| 2 | 3.4 |

TABLE 4
Maximum concentration of hydromorphone (HM) found
in blood after oral (PO) dosing in rats.

| Example | Cmax HM (ng/ml) following PO dosing |
|---|---|
| Hydromorphone | 45 |
| 1 | 7.2 |

Compared to hydromorphone, compounds according to the invention afford a lower Cmax of hydromorphone when administered IV, but demonstrate similar or attenuated Cmax values to hydromorphone when administered orally.

TABLE 5
Maximum concentration (Cmax) of hydrocodone
(HC) found in blood after IV dosing in rats.

| Example | Cmax HC (ng/ml) following IV dosing |
|---|---|
| 11 | 90 |
| 13 | ND |

ND = not detected

TABLE 6
Maximum concentration of hydrocodone (HC) found
in blood after oral (PO) dosing in rats.

| Example | Dose (mg/kg) | Cmax HC (ng/ml) following PO dosing |
|---|---|---|
| Hydrocodone | 20 | 23.6 |
| 6 | 15 | ND |
| 6 | 30 | 6.0 |
| 7 | 20 | 1.2 |
| 8 | 15 | ND |
| 9 | 15 | ND |
| 10 | 20 | ND |
| 11 | 20 | 3.8 |
| 12 | 20 | 1.2 |
| 13 | 20 | 2.7 |

ND = not detected

The data presented in Table 5 indicated that tested compounds of Examples 11 and 13 afford a low to undetectable Cmax of hydrocodone when administered IV.

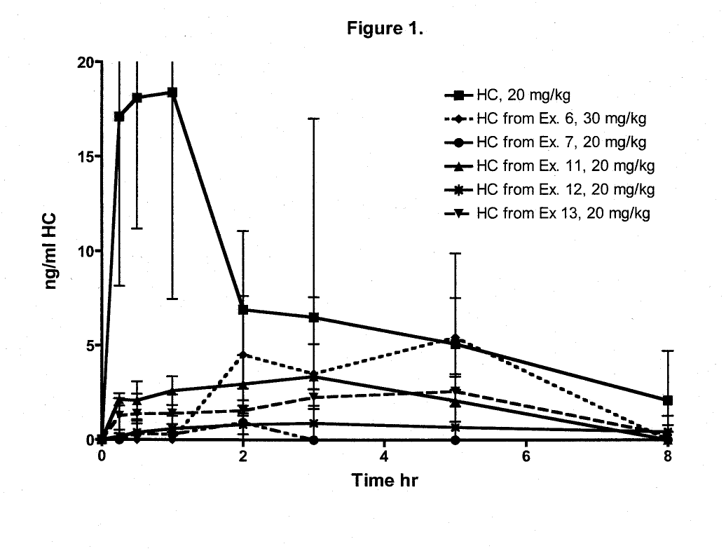

The data presented in Table 6 and FIG. 1 demonstrate that, compared to hydrocodone, tested compounds of Examples 6 (at the 30 mg/kg dose), 7, 11, 12 and 13 demonstrate attenuated Cmax values of hydrocodone when administered orally. The doses used for the test compounds of Examples 6 (at the 15 mg/ml dose), 8, 9, and 10 did not afford detectable levels of HC, but it is not believed that this result indicates that these test compounds are incapable of functioning as prodrugs for HC. These doses may have been too low for the specific model and/or analytical methods employed; the undetectable Cmax value for the test compound of Example 6 at 15 mg/kg compared to the Cmax value for the 30 mg/kg dose exemplifies this conclusion. It should be noted that the test compounds were dosed as mg/kg body weight, not mg equivalents of HC.

1. A compound of general formula:
R₁R₂R₃N⁺—(C(R^{1a})(R^{2a}))—Ar—Z—C(O)—Y—(C(R¹)(R²))ₙ—N—(R³)(R⁴)A⁻ II
or a salt, hydrate or solvate thereof wherein:
R₁R₂R₃N⁺—represents a residue of an opioid wherein the lone pair of electrons of the tertiary amine nitrogen atom is replaced with a bond to —(C(R^{1a})(R^{2a})—Ar—Z—C(O)—Y—(C(R¹)(R²))ₙ—N—(R³)(R⁴);
R^{1a} and R^{2a} are independently hydrogen, alkyl, substituted alkyl, alkoxy, substituted alkoxy, aryl, substituted aryl, arylalkyl, substituted arylalkyl, heteroaryl, substituted heteroaryl, heteroarylalkyl or substituted heteroarylalkyl;
Ar is aryl, heteroaryl or arylaryl optionally substituted with one or more —F, —Cl, —Br, —I, —R^{4a}, —O⁻, —OR^{4a}, —SR^{4a}, —S⁻, —NR^{4a}R^{5a}, —CF₃, —CN, —OCN, —SCN, —NO, —NO₂, —N₃, —S(O)₂O⁻, —S(O)₂OH, —S(O)₂R^{4a}, —OS(O₂)O⁻, —OS(O)₂R^{4a}, —P(O)(O)₂, —P(O)(OR^{4a})(O⁻), —OP(O)(OR^{4a})(OR^{5a}), —C(O)R^{4a}, —C(S)R^{4a}, —C(O)OR^{4a}, —C(O)NR^{4a}R^{5a}, —C(O)O⁻, —C(S)OR^{4a}, —NR^{6a}C(O)NR^{4a}R^{5a}, —NR^{6a}C(S)NR^{4a}R^{5a}, —NR^{7a}C(NR^{6a})NR^{5a}R^{4a} or —C(NR^{6a})NR^{5a}R^{4a}, or tethered to a polymer;
R^{4a}, R^{5a}, R^{6a} and R^{7a} are independently hydrogen, alkyl, substituted alkyl, cycloalkyl, substituted cycloalkyl, cycloheteroalkyl, substituted cycloheteroalkyl, aryl, substituted aryl, heteroaryl or substituted heteroaryl, or optionally R⁴ and R⁵ together with the nitrogen atom to which they are bonded form a cycloheteroalkyl or substituted cycloheteroalkyl ring;
Z is O, S or NH;
Y is —NR⁵—, —O— or —S—;
n is an integer from 1 to 10;
each R¹, R², R³ and R⁵ is independently hydrogen, alkyl, substituted alkyl, aryl or substituted aryl, or R¹ and R² together with the carbon to which they are attached form a cycloalkyl or substituted cycloalkyl group, or two R¹ or R² groups on adjacent carbon atoms, together with the carbon atoms to which they are attached, form a cycloalkyl or substituted cycloalkyl group; 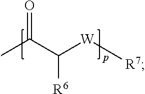
each R⁶ is independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl, or optionally, R⁶ and R⁷ together with the atoms to which they are bonded form a cycloheteroalkyl or substituted cycloheteroalkyl ring;
R⁷ is hydrogen, alkyl, substituted alkyl, acyl, substituted acyl, alkoxycarbonyl, substituted alkoxycarbonyl, aryl, substituted aryl, arylalkyl or substituted arylalkyl;
p is an integer from 1 to 5;
each W is independently —NR⁸—, —O— or —S—;
each R⁸ is independently hydrogen, alkyl, substituted alkyl, aryl or substituted aryl, or optionally, each R⁶ and R⁸ independently together with the atoms to which they are bonded form a cycloheteroalkyl or substituted cycloheteroalkyl ring; and
A⁻ represents an anion.

2. The compound of claim 1, in which R₁R₂R₃N⁺— is a residue of hydrocodone, oxycodone, hydromorphone or oxymorphone.

3. The compound of claim 1, in which R₁R₂R₃N⁺— is a residue of hydrocodone, hydromorphone or oxymorphone.

4. The compound of claim 1, in which R^{1a} and R^{2a} each represents hydrogen.

5. The compound of claim 1, in which Ar represents a 1,2-phenylene or 1,4-phenylene group that is unsubstituted or substituted by one or two substituents selected independently from a halogen atom; a (1-4C)alkyl group; a (1-4C)alkoxy group; a carboxy group; and a hydroxy(1-4C)alkyl group.

6. The compound of claim 5, in which Ar represents 1,4-phenylene.

7. The compound of claim 1, in which Z represents O.

8. The compound of claim 1, in which Y is NR⁵ and R⁵ is hydrogen or alkyl.

9. The compound of claim 1, in which n is 2 or 3.

10. The compound of claim 1, in which R¹, R², R³, R⁵ and R⁸ are independently hydrogen or alkyl.

11. The compound of claim 1, in which each R⁶ is independently hydrogen, alkyl, substituted alkyl, aryl, arylalkyl, substituted arylalkyl, heteroalkyl, heteroarylalkyl, substituted heteroarylalkyl, or optionally, R⁶ and R⁷ together with the atoms to which they are bonded form a cycloheteroalkyl or substituted cycloheteroalkyl ring.

12. The compound of claim 1, in which R⁷ is hydrogen, alkyl, acyl or alkoxycarbonyl.

13. The compound of claim 1, in which Y is NR⁵, n is 2 or 3, p is 1 or 2, R¹, R², R³, R⁵ and R⁷ are independently hydrogen or alkyl, each R⁶ is independently hydrogen, alkyl, substituted alkyl, aryl, arylalkyl, substituted arylalkyl, heteroalkyl, heteroarylalkyl, substituted heteroarylalkyl or optionally, R⁶ and R⁷ together with the atoms to which they are bonded form a cycloheteroalkyl or substituted cycloheteroalkyl ring.

14. The compound of claim 1, in which Y is NR⁵, n is 2, p is 1, R¹ and R² are hydrogen, R³ and R⁵ are independently methyl or hydrogen and R⁶ is independently hydrogen, alkyl, substituted alkyl, aryl, arylalkyl, substituted arylalkyl, heteroalkyl, heteroarylalkyl, substituted heteroarylalkyl or optionally, R⁶ and R⁷ together with the atoms to which they are bonded form a cycloheteroalkyl or substituted cycloheteroalkyl ring or optionally R⁷ is hydrogen.

15. The compound of claim 1, in which Y is NR⁵, n is 2, R¹ and R² are hydrogen, R³ and R⁵ are independently methyl or hydrogen, R⁷ is hydrogen and R⁶ is —CH₂(CH₂)₃NH₂ or —CH₂CH₂CH₂NHC(NH)NH₂.

16. The compound of claim 1, in which Y is —NR⁵; R⁵ is (1-4C)alkyl;
R¹ and R² are each hydrogen; n is 2 or 3; R³ is hydrogen or (1-4C)alkyl; W is NH;
R⁶ is H, —CH₃, —CH₂CH(CH₃)₂, —CH₂(CH₂)₃NH₂, —CH₂CH₂CH₂NHC(NH)NH₂, —CH₂C(═O)NH₂, —CH₂COOH, —CH₂ (p-hydroxyphenyl) or CH₂CH₂COOH;
R⁷ is hydrogen, (1-6C)alkanoyl or benzoyl unsubstituted or substituted by methylenedioxy or one or two substituents selected from (1-4C)alkyl, (1-4C)alkoxy and halogen; and p is 1 or 2.

17. The compound of claim 1, in which Y is —NR⁵; R⁵ is (1-4C)alkyl;
R¹ and R² are each hydrogen; n is 2 or 3; R³ is hydrogen or (1-4C)alkyl; W is NH;
R⁶ is hydrogen, —CH₂ (CH₂)₃NH₂, —CH₂CH₂CH₂NHC(NH)NH₂ or CH₂CH₂COOH;
R⁷ is hydrogen, (1-6C)alkanoyl or benzoyl unsubstituted or substituted by methylenedioxy or one or two substituents selected from (1-4C)alkyl, (1-4C)alkoxy and halogen; and p is 1 or 2.

18. The compound of claim 1, in which R⁴ is a residue of a D or L-amino acid selected from alanine, arginine, asparagine, aspartic acid, cysteine, glycine, glutamine, glutamic acid, histidine, isoleucine, leucine, methionine, phenylalanine, proline, serine, threonine, tryptophan, tyrosine, lysine and valine; a residue of a dipeptide or tripeptide composed of two or three L-amino acid residues selected independently from alanine, arginine, asparagine, aspartic acid, cysteine, glycine, glutamine, glutamic acid, histidine, isoleucine, leucine, methionine, phenylalanine, proline, serine, threonine, tryptophan, tyrosine, lysine and valine; or a residue of an N-acyl derivative thereof.

19. The compound of claim 18, which is a residue of an L-amino acid.

20. The compound of claim 18, in which R⁴ is arginine, N-acetylarginine, N-t-butanoylarginine, N-benzoylarginine, N-piperonylarginine, N-glycinylarginine, N-acetylglycinylarginine, alanine, N-acetylalanine, asparagine, N-acetylasparagine, aspartic acid, N-acetylaspartic acid, lysine, N-acetyllysine, leucine, N-acetylleucine, glutamic acid, tyrosine, N-acetyltyrosine, proline or N-glycinylproline.

21. The compound of claim 18, in which R⁴ is arginine, N-acetylarginine, N-t-butanoylarginine, N-benzoylarginine, N-piperonylarginine, N-glycinylarginine, lysine, glutamic acid, proline or N-glycinylproline.

22. The compound of claim 1, which is selected from pharmaceutically acceptable salts of:
hydrocodone N-(4-(N′-methyl-N′-(2-L-leucinylaminoethyl)aminocarbonyloxy)phenylmethyl;
hydrocodone N-(4-(N′-methyl-N′-(2-arginylaminoethyl)aminocarbonyloxy)phenylmethyl;
hydrocodone N-(4-(N′-methyl-N′-(2-lysinylaminoethyl)aminocarbonyloxy)phenylmethyl;
hydrocodone N-(4-(N′-methyl-N′-(2-glycinylarginylaminoethyl)aminocarbonyloxy)-phenylmethyl; and
hydrocodone N-(4-(N′-methyl-N′-(2-L-asparaginylaminoethyl)aminocarbonyloxy)phenylmethyl.

23. A process for the preparation of a compound as claimed in claim 1, which comprises reacting a compound of formula (V)
M¹-(C(R^{1a})(R^{2a}))—Ar—Z—C(O)—Y—(C(R¹)(R²))ₙ—N—(R³)(R⁴) (V)
or a protected derivative thereof, in which M¹ represents a leaving atom or group, such as a chlorine atom, with an opioid, followed by removing any protecting groups and, if desired, acylating a compound of formula (II) in which R⁷ (in the group R⁴ as defined hereinabove) represents a hydrogen atom and/or forming a pharmaceutically acceptable salt.

24. A pharmaceutical composition, which comprises a compound as claimed in claim 1 and a pharmaceutically acceptable vehicle.

25. A method of providing a patient with post administration-activated, controlled release of an opioid, which comprises administering to said patient a compound as claimed in claim 1.

26. (canceled)

27. (canceled)

28. (canceled)

29. The compound of claim 1, in which R₁R₂R₃N⁺— is a residue of hydrocodone.

30. The compound of claim 1, in which R⁴ is arginine, N-acetylarginine, lysine, or N-acetyllysine.

31. The compound of claim 1, wherein
R₁R₂R₃N⁺— represents a residue of an opioid selected from hydrocodone, oxycodone, oxymorphone, and hydromorphone residue;
R^{1a} and R^{2a} is hydrogen;
Ar is 1,4-phenylene;
Z is O;
Y is —NR⁵;
R⁵ is (1-4C)alkyl;
R¹ and R² are independently selected from hydrogen and (1-4C)alkyl;
n is 2 or 3;
R³ is hydrogen or (1-4C)alkyl;
R⁴ is selected from arginine, N-acetylarginine, N-t-butanoylarginine, N-benzoylarginine, N-piperonylarginine, N-glycinylarginine, N-acetylglycinylarginine, alanine, N-acetylalanine, asparagine, N-acetylasparagine, aspartic acid, N-acetylaspartic acid, lysine, N-acetyllysine, leucine, N-acetylleucine, glutamic acid, tyrosine, N-acetyltyrosine, proline and N-glycinylproline;
A⁻ is Cl⁻.

32. The compound of claim 31, wherein R⁴ is selected from arginine, N-acetylarginine, N-t-butanoylarginine, N-benzoylarginine, N-piperonylarginine, N-glycinylarginine, lysine, glutamic acid, proline and N-glycinylproline.
